(19) United States
(12) Patent Application Publication
(10) Pub. No.: US 2008/0009467 A1
(43) Pub. Date: Jan. 10, 2008

(54) COMBINATIONS OF MEDIUM CHAIN TRIGLYCERIDES AND THERAPEUTIC AGENTS FOR THE TREATMENT AND PREVENTION OF ALZHEIMERS DISEASE AND OTHER DISEASES RESULTING FROM REDUCED NEURONAL METABOLISM

(75) Inventor: Samuel T. Henderson, Broomfield, CO (US)

Correspondence Address: SWANSON & BRATSCHUN, L.L.C., 8210 SOUTHPARK TERRACE, LITTLETON, CO, 80120, US

(73) Assignee: ACCERA, INC., Broomfield, CO (US)

(21) Appl. No.: 11/771,431

(22) Filed: Jun. 29, 2007

Related U.S. Application Data
(63) Continuation-in-part of application No. 11/021,920, filed on Dec. 22, 2004.
Application No. 11/021,920 is a continuation of application No. 10/152,147, filed on May 20, 2002, now Pat. No. 6,835,750.
Application No. 10/152,147 is a continuation-in-part of application No. 09/845,741, filed on May 1, 2001, now abandoned.
(60) Provisional application No. 60/200,980, filed on May 1, 2000.

(51) Int. Cl. A61K 31/225; A61K 31/407; A61K 31/435; A61K 31/445; A61K 31/55; A61K 31/662; A61K 31/7004; A61P 25/28

(52) U.S. Cl. 514/129; 514/215; 514/25; 514/295; 514/297; 514/319; 514/412; 514/547

(57) ABSTRACT

Methods and compositions for treating or preventing, the occurrence of senile dementia of the Alzheimer's type, mild cognitive impairment, or other conditions arising from reduced neuronal metabolism and leading to lessened cognitive function are described. In a preferred embodiment the administration of triglycerides or fatty acids with chain lengths between 5 and 12, to said patient at a level to produce an improvement in cognitive ability, and a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof. Preferred therapeutic agents include donepezil, rivastigmine, galantamine, and memantine.

### CROSS REFERENCE TO RELATED APPLICATIONS

This application is a continuation-in-part of U.S. application Ser. No. 11/021,920, filed Dec. 22, 2004, entitled “Use of Medium Chain Trigylcerides for the Treatment and Prevention of Alzheimer's Disease and Other Diseases Resulting from Reduced Neuronal Metabolism II,” which is a continuation of Ser. No. 10/152,147, filed May 20, 2002, entitled “Use of Medium Chain Trigylcerides for the Treatment and Prevention of Alzheimer's Disease and Other Diseases Resulting from Reduced Neuronal Metabolism II,” now U.S. Pat. No. 6,835,750, which is a continuation in part of U.S. application Ser. No. 09/845,741, filed May 1, 2001, which claims priority to U.S. Provisional Application Ser. No. 60/200,980 filed May 1, 2000, entitled “Use of Medium Chain Triglycerides for the Treatment and Prevention of Alzheimer's Disease and Other Diseases Resulting from Reduced Neuronal Metabolism.” This application also claims the benefit of U.S. application Ser. No. 11/611,114, filed Dec. 14, 2006, entitled “Compositions And Methods For Improving Or Preserving Brain Function.”

### FIELD OF THE INVENTION

This invention relates to the field of therapeutic agents for the treatment of Alzheimer's disease, Mild Cognitive Impairment, and other diseases associated with reduced neuronal metabolism, including Parkinson's disease, Huntington's Disease, and epilepsy.

### BACKGROUND OF THE INVENTION

Alzheimer's disease (AD) is a progressive neurodegenerative disorder, which primarily affects the elderly. There are two forms of AD, early-onset and late-onset. Early-onset AD is rare, strikes susceptible individuals as early as the third decade, and is frequently associated with mutations in a small set of genes. Late onset, or spontaneous, AD is common, strikes in the seventh or eighth decade, and is a mutifactorial disease with many genetic risk factors. Late-onset AD is the leading cause of dementia in persons over the age of 65. An estimated 7-10% of the American population over 65, and up to 40% of the American population greater than 80 years of age is afflicted with AD (McKhann et al., 1984; Evans et al. 1989). Early in the disease, patients experience loss of memory and orientation. As the disease progresses, additional cognitive functions become impaired, until the patient is completely incapacitated. Many theories have been proposed to describe the chain of events that give rise to AD, yet, at the time of this application, the cause remains unknown. Currently, no effective prevention or treatment exists for AD. Drugs to treat AD on the market today, Aricept®, Cognex®, Reminyl®/Razadyne®, Exelon® and Namenda® do not address the underlying pathology of AD. They merely enhance the effectiveness of those nerve cells still able to function and only provide symptomatic relief from the disease.

Metabolism and Alzheimer's disease. At the time of this application, the cause of AD remains unknown, yet a large body of evidence has made it clear that Alzheimer's disease is associated with decreased neuronal metabolism. In 1984, Blass and Zemcov proposed that AD results from a decreased metabolic rate in sub-populations of cholinergic neurons. However, it has become clear that AD is not restricted to cholinergic systems, but involves many types of transmitter systems, and several discrete brain regions. Positron-emission tomography has revealed poor glucose utilization in the brains of AD patients, and this disturbed metabolism can be detected well before clinical signs of dementia occur (Reiman et al., 1996; Messier and Gagnon, 1996; Hoyer, 1998). Additionally, certain populations of cells, such as somatostatin cells of the cortex in AD brain are smaller, and have reduced Golgi apparatus; both indicating decreased metabolic activity (for review see Swaab et al. 1998). Measurements of the cerebral metabolic rates in healthy versus AD patients demonstrated a 20-40% reduction in glucose metabolism in AD patients (Hoyer, 1992). Reduced glucose metabolism results in critically low levels of ATP in AD patients. Also, the severity of decreased metabolism was found to correlate with senile plaque density (Meier-Ruge, et al. 1994).

Additionally, molecular components of insulin signaling and glucose utilization are impaired in AD patients. Glucose is transported across the blood brain barrier and is used as a major fuel source in the adult brain. Consistent with the high level of glucose utilization, the brains of mammals are well supplied with receptors for insulin and IGF, especially in the areas of the cortex and hippocampus, which are important for learning and memory (Frolich et al., 1998). In patients diagnosed with AD, increased densities of insulin receptor were observed in many brain regions, yet the level of tyrosine kinase activity that normally is associated with the insulin receptor was decreased, both relative to age-matched controls (Frolich et al., 1998). The increased density of receptors represents up-regulation of receptor levels to compensate for decreased receptor activity. Activation of the insulin receptor is known to stimulate phosphatidylinositol-3 kinase (PI3K). PI3K activity is reduced in AD patients (Jolles et al., 1992; Zubenko et al., 1999). Furthermore, the density of the major glucose transporters in the brain, GLUT1 and GLUT3, was found to be 50% of age matched controls (Simpson and Davies, 1994). The disturbed glucose metabolism in AD has led to the suggestion that AD may be a form of insulin resistance in the brain, similar to type II diabetes (Hoyer, 1998). Inhibition of insulin receptor activity can be exogenously induced in the brains of rats by intracerebroventricular injection of streptozotocin, a known inhibitor of the insulin receptor. These animals develop progressive defects in learning and memory (Lannert and Hoyer, 1998). While glucose utilization is impaired in brains of AD patients, use of the ketone bodies, beta-hydroxybutyrate and acteoacetate is unaffected (Ogawa et al., 1996).

The cause of decreased neuronal metabolism in AD remains unknown. Yet, aging may exacerbate the decreased glucose metabolism in AD. Insulin stimulation of glucose uptake is impaired in the elderly, leading to decreased insulin action and increased insulin resistance (for review see Finch and Cohen, 1997). For example, after a glucose load, mean plasma glucose is 10-30% higher in those over 65 than in younger subjects. Hence, genetic risk factors for AD may result in slightly compromised neuronal metabolism in the brain. These defects would only become apparent later in life when glucose metabolism becomes impaired, and thereby contribute to the development of AD. Since the defects in glucose utilization are limited to the brain in AD, the liver is “unaware” of the state of the brain and does not mobilize fatty acids (see Brain Metabolism section below). Without ketone bodies to use as an energy source, the neurons of the AD patient brain slowly and inexorably starve to death.

Attempts to compensate for reduced cerebral metabolic rates in AD patients has met with some success. Treatment of AD patients with high doses of glucose and insulin increases cognitive scores (Craft et al., 1996). However, since insulin is a polypeptide and must be transported across the blood brain barrier, delivery to the brain is complicated. Therefore, insulin is administered systemically. A large dose of insulin in the blood stream can lead to hyperinsulinemia, which will cause irregularities in other tissues. Both of these shortcomings make this type of therapy difficult and rife with complications. Accordingly, there remains a need for an agent that may increase the cerebral metabolic rate and subsequently the cognitive abilities of a patient suffering from Alzheimer's disease.

Brain Metabolism. The brain has a very high metabolic rate. For example, it uses 20 percent of the total oxygen consumed in a resting state. Large amounts of ATP are required by neurons of the brain for general cellular functions, maintenance of an electric potential, synthesis of neurotransmitters and synaptic remodeling. Current models propose that under normal physiologic conditions, neurons of the adult human brain depend solely on glucose for energy. Since neurons lack glycogen stores, the brain depends on a continuous supply of glucose from the blood for proper function. Neurons are very specialized and can only efficiently metabolize a few substrates, such as glucose and ketone bodies. This limited metabolic ability makes brain neurons especially vulnerable to changes in energy substrates. Hence, sudden interruption of glucose delivery to the brain results in neuronal damage. Yet, if glucose levels drop gradually, such as during fasting, neurons will begin to metabolize ketone bodies instead of glucose and no neuronal damage will occur.

Neuronal support cells, glial cells, are much more metabolically diverse and can metabolize many substrates, in particular, glial cells are able to utilize fatty acids for cellular respiration. Neurons of the brain cannot efficiently oxidize fatty acids and hence rely on other cells, such as liver cells and astrocytes to oxidize fatty acids and produce ketone bodies. Ketone bodies are produced from the incomplete oxidation of fatty acids and are used to distribute energy throughout the body when glucose levels are low. In a normal Western diet, rich in carbohydrates, insulin levels are high and fatty acids are not utilized for fuel, hence blood ketone body levels are very low, and fat is stored and not used.

Current models propose that only during special states, such as neonatal development and periods of starvation, will the brain utilize ketone bodies for fuel. The partial oxidation of fatty acids gives rise to D-beta-hydroxybutyrate (D-3-β-hydroxybutyrate) and acetoacetate, which together with acetone are collectively called ketone bodies. Neonatal mammals are dependent upon milk for development. The major carbon source in milk is fat (carbohydrates make up less then 12% of the caloric content of milk). The fatty acids in milk are oxidized to give rise to ketone bodies, which then diffuse into the blood to provide an energy source for development. Numerous studies have shown that the preferred substrates for respiration in the developing mammalian neonatal brain are ketone bodies. Consistent with this observation is the biochemical finding that astrocytes, oligodendrocytes and neurons all have capacity for efficient ketone body metabolism (for review see Edmond, 1992). Yet only astrocytes are capable of efficient oxidation of fatty acids to ketone bodies.

The body normally produces small amounts of ketone bodies. However, because they are rapidly utilized, the concentration of ketone bodies in the blood is very low. Blood ketone body concentrations rise on a low carbohydrate diet, during periods of fasting, and in diabetics. In a low carbohydrate diet, blood glucose levels are low, and pancreatic insulin secretion is not stimulated. This triggers the oxidation of fatty acids for use as a fuel source when glucose is limiting. Similarly, during fasting or starvation, liver glycogen stores are quickly depleted, and fat is mobilized in the form of ketone bodies. Since both a low carbohydrate diet and fasting do not result in a rapid drop of blood glucose levels, the body has time to increase blood ketone levels. The rise in blood ketone bodies provides the brain with an alternative fuel source, and no cellular damage occurs. Since the brain has such high energy demands, the liver oxidizes large amounts of fatty acids until the body becomes literally saturated with ketone bodies. Therefore, when an insufficient source of ketone bodies is coupled with poor glucose utilization severe damage to neurons results. Since glial cells are able to utilize a large variety of substrates they are less susceptible to defects in glucose metabolism than are neurons. This is consistent with the observation that glial cells do not degenerate and die in AD (Mattson, 1998).

As discussed in the Metabolism and Alzheimer's disease section, in AD, neurons of the brain are unable to utilize glucose and begin to starve. Since the defects are limited to the brain and peripheral glucose metabolism is normal, the body does not increase production of ketone bodies, therefore neurons of the brain slowly starve to death. Accordingly, there remains a need for an energy source for brain cells that exhibit compromised glucose metabolism. Compromised glucose metabolism is a hallmark of AD; hence administration of such an agent will prove beneficial to those suffering from AD.

Medium Chain Triglycerides (MCT). The metabolism of MCT differs from the more common long chain triglycerides (LCT) due to the physical properties of MCT and their corresponding medium chain fatty acids (MCFA). Due to the short chain length of MCFA, they have lower melting temperatures, for example the melting point of MCFA (C8:0) is 16.7° C., compared with 61.1° C. for the LCFA (C16:0). Hence, MCT and MCFA are liquid at room temperature. MCT are highly ionized at physiological pH, thus they have much greater solubility in aqueous solutions than LCT. The enhanced solubility and small size of MCT also increases the rate at which fine emulsion particles are formed. These small emulsion particles create increased surface area for action by gastrointestinal lipases. Additionally, medium chain 2-monoglycerides isomerize more rapidly than those of long chain length, allowing for more rapid hydrolysis. Some lipases in the pre-duodenum preferentially hydrolyze MCT to MCFA, which are then partly absorbed directly by stomach mucosa (Hamosh, 1990). Those MCFA which are not absorbed in the stomach, are absorbed directly into the portal vein and not packaged into lipoproteins. LCFA are packaged in chylomicrons and transported via the lymph system, while MCFA are transported via the blood. Since blood transports much more rapidly than lymph, the liver is quickly perfused with MCFA. MCFA enter the mitochondria largely without the use of carnitine palmitoyltransferase I, therefore MCFA by-pass this regulatory step and are oxidized regardless of the metabolic state of the organism. Importantly, since MCFA enter the liver rapidly and are quickly oxidized, large amounts of ketone bodies are readily produced from MCFA.

Numerous patents relate to use of MCT. None of these patents relate to the specific use of MCT for treatment and prevention of Alzheimer's disease or other neurodegenerative diseases. Patents such as U.S. Pat. No. 4,528,197 “Controlled triglyceride nutrition for hypercatabolic mammals” and U.S. Pat. No. 4,847,296 “Triglyceride preparations for the prevention of catabolism” relate to the use of MCT to prevent body-wide catabolism that occurs in burns and other serious injuries. Each patent described herein anywhere in the current specification is incorporated by reference herein in its entirety.

A need remains in the art for treatments that may prevent, treat, or ameliorate the symptoms of Alzheimer's disease, mild cognitive impairment, and other diseases of reduced neuronal metabolism.

### SUMMARY OF THE INVENTION

In one embodiment, the present invention provides a composition for the treatment of or prevention of Alzheimer's disease or mild cognitive impairment. The composition comprises medium chain triglycerides (MCT) of the formula: 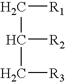
wherein the R1, R2, and R3 esterified to the glycerol backbone are each independently fatty acids having 5-12 carbon chains in an amount effective for the treatment of or prevention of loss of cognitive function in a mammal caused by reduced neuronal metabolism in dementia of Alzheimer's type or mild cognitive impairment. The composition also comprises at least one therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof.

In another embodiment, the present invention includes a method of treating dementia of Alzheimer's type or mild cognitive impairment. This method includes the step of identifying a mammal having, or at risk of dementia of Alzheimer's type or mild cognitive impairment. The method further comprises administering to the mammal a first composition comprising medium chain triglycerides (MCT) of the formula: 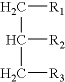
wherein the R1, R2, and R3 esterified to the glycerol backbone are each independently fatty acids having 5-12 carbon chains in an amount effective for the treatment of or prevention of loss of cognitive function caused by reduced neuronal metabolism in dementia of Alzheimer's type or mild cognitive impairment; and administering a second composition comprising at least one therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof.

In one embodiment, the anti-Alzheimer's agent can be at least one of the following: modulators of cholinesterase, acetylcholine synthesis modulators, acetylcholine storage modulators, acetylcholine release modulators, NMDA receptor antagonists, beta-amyloid inhibitors, β-amyloid plaque removal agents (including vaccines), inhibitors of β-amyloid plaque formation, amyloid precursor protein processing enzyme inhibitors, β-amyloid converting enzyme (BACE) inhibitors, β-secretase inhibitors, γ-secretase modulators, nerve growth factor agonists, hormone receptor blockade agents, neurotransmission modulators, anti-inflammatory agents, and combinations thereof.

In one embodiment, the invention includes a liquid dosage form for oral consumption. This liquid dosage form includes a unit dose of MCT sufficient to a) raise blood levels of D-β-hydroxybutyrate to about 0.1 to about 5 mM or b) raise urinary excretion levels of D-β-hydroxybutyrate to about 5 mg/dL to about 160 mg/dL; a plurality of vitamins; flavoring, and a carbohydrate source and wherein the MCT are of the formula:

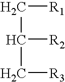

wherein the R1, R2, and R3 esterified to the glycerol backbone are each independently fatty acids having carbon chains of 5-12 carbons. This liquid oral dosage form also includes a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof.

### DETAILED DESCRIPTION OF THE INVENTION

It is the novel insight of this invention that compositions comprising at least one compound capable of elevating ketone body concentrations, such as, for example, medium chain triglycerides (MCT) and/or their associated medium chain fatty acids, and further comprising a therapeutic agent such as, for example, an anti-Alzheimer's agent, an anti-diabetic agent, an agent capable of increasing utilization of lipids, an anti-atherosclerotic agent, an anti-hypertensive agent, an anti-inflammatory agent, an anti-obesity agent, and combinations thereof, are useful as a treatment and preventative measure for diseases of reduced neuronal metabolism, such as Alzheimer's disease and mild cognitive impairment. Synergistic effects from the combination therapy were noted. As used herein, “patient” refers to any mammal, including humans, that may benefit from treatment of disease and conditions resulting from reduced neuronal metabolism. As used herein, reduced neuronal metabolism refers to all possible mechanisms that could lead to a reduction in neuronal metabolism. Such mechanisms include, but are not limited to mitochondrial dysfunction, free radical attack, defective glucose transport or glycolysis, imbalance in membrane ionic potential, dysfunction in calcium flux, and the like. MCT are composed of fatty acids with chain lengths of between 5-12 carbons. A diet rich in MCT and/or an MCT precursor results in high blood ketone levels. High blood ketone levels provide an energy source for brain cells that have compromised glucose metabolism via the rapid oxidation of MCFA to ketone bodies.

In light of the deficiencies in other methods to treat deficits of energy metabolism in the brain, the present invention contemplates use of another substance to improve memory performance, in particular, ketone bodies, which are is known to be readily utilized by the brain. Co-administration of a compound which is capable of elevating ketone body concentrations with a therapeutic agent such as, for example, an anti-Alzheimer's agent, an anti-diabetic agent, an agent capable of increasing utilization of lipids, an anti-atherosclerotic agent, an anti-hypertensive agent, an anti-inflammatory agent, an anti-obesity agent, and combinations thereof, is a novel and are useful as a treatment and preventative measure for diseases of reduced neuronal metabolism, such as Alzheimer's disease and mild cognitive impairment. Synergistic effects from the combination therapy were noted.

Ketone bodies, in particular β-hydroxybutyrate, (βHB) and acetoacetate serve a critical role in the development and health of cerebral neurons. Numerous studies have shown that the preferred substrates for the developing mammalian neonatal brain are ketone bodies. There is a large body of evidence demonstrating that ketone bodies are used in a concentration dependent manner, even in the elderly. Ketone bodies (KB) offer several advantages to glucose for memory facilitation in the elderly. (1) KB can be artificially elevated by the administration of large amounts of medium chain triglycerides (MCT) or other substance capable of raising ketone body levels without altering glucose levels. (2) Hyperketonemia can be induced and sustained for many hours. (3) KB readily cross the blood brain barrier. (4) KB are readily metabolized by cerebral neurons and can be used to generate ATP and acetylcholine. In particular, a composition developed by the inventors, KETASYN, in conjunction with an anti-Alzheimer's agent, an anti-diabetic agent, an agent capable of increasing utilization of lipids, an anti-atherosclerotic agent, an anti-hypertensive agent, an anti-inflammatory agent, an anti-obesity agent, and combinations thereof, provides a simple and safe method to induce hyperketonemia and takes advantage of synergistic benefits of MCT administration in conjunction together with one or more therapeutic agents.

MCT are comprised of fatty acids with a chain length of between 5-12 carbons and have been researched extensively. MCT are metabolized differently from the more common long chain triglycerides (LCT). In particular, when compared to LCT, MCT are more readily digested to release medium chain fatty acids (MCFA) which exhibit increased rates of portal absorption, and undergo obligate oxidation. MCFA have melting points much lower than long chain fatty acids (LCFA), and therefore the MCFA and corresponding MCT are liquid at room temperature. MCFA are smaller and more ionized at physiological pH compared to LCFA, and hence MCFA are much more soluble in aqueous solutions. The small size and decreased hydrophobicity of MCT increases the rate of digestion and absorption relative to LCT.

When ingested, MCT are first processed by lipases, which cleave the fatty acid chains from the glycerol backbone. Some lipases in the pre-duodenum preferentially hydrolyze MCT over LCT and the released MCFA are then partly absorbed directly by the stomach mucosa. Those MCFA which are not absorbed in the stomach are absorbed directly into the portal vein and are not packaged into lipoproteins. LCFA derived from normal dietary fat are re-esterified into LCT and packaged into chylomicrons for transport in the lymph. This greatly slows the metabolism of LCT relative to MCT. Since blood transports much more rapidly than lymph, MCFA quickly arrive at the liver.

In the liver MCFA undergo obligate oxidation. In the fed state LCFA undergo little oxidation in the liver, due mainly to the inhibitor effects of malonyl-CoA. When conditions favor fat storage, malonyl-CoA is produced as an intermediate in lipogenesis. Malonyl-CoA allosterically inhibits carnitine palmitoyltransferase I, and thereby inhibits LCFA transport into the mitochondria. This feedback mechanism prevents futile cycles of lipolysis and lipogenesis. MCFA are, to a large extent, immune to the regulations that control the oxidation of LCFA. MCFA enter the mitochondria without the use of carnitine palmitoyltransferase I, therefore MCFA bypass this regulatory step and are oxidized regardless of the metabolic state of the organism. Importantly, since MCFA enter the liver rapidly and are quickly oxidized, large amounts of ketone bodies are readily produced from MCFA and a large oral dose of MCT (roughly 20 mL) will result in sustained hyperketonemia. It is the novel insight of the inventor that MCT may be administered outside of the context of a ketogenic diet (e.g. a low carbohydrate diet). Therefore, in the present invention carbohydrates may be consumed at the same time as MCT. This represents a significant advantage over the prior art, which only describes the use of MCT in the context of a ketogenic diet. Such diets greatly restrict both carbohydrate and protein in the diet, and are, in practice, extremely difficult for patients to comply with. The present invention represents a significant advantage over ketogenic diets involving low carbohydrate intake, in that the present invention, the subject is free to follow any diet and does not have to adhere to any dietary restrictions.

According to the present invention, high blood ketone levels will provide an energy source for brain cells that have compromised glucose metabolism, via the rapid oxidation of MCFA to ketone bodies, leading to improved performance in, and/or reversal, prevention, reduction and/or delaying of decline in Alzheimer's disease, mild cognitive impairment, or a parameter indicative of Alzheimer's disease or mild cognitive impairment, e.g., ADAS-cog, MMSE, Stroop Color Word Interference Task, Logical Memory subtest of the Wechsler Memory Scale-III, Clinician's Dementia Rating, and Clinician's Interview Based Impression of Change.

Various terms relating to the methods and other aspects of the present invention are used throughout the specification and claims; such terms are to be given their ordinary meaning in the art unless otherwise indicated. Other specifically defined terms are to be construed in a manner consistent with the definition provided herein.

The background of this invention supports the present invention in the following ways. (1) Neurons of the brain can use both glucose and ketone bodies for respiration. (2) The neurons of Alzheimer's disease and/or mild cognitive impairment patients have well documented defects in glucose metabolism. (3) Known genetic risk factors for Alzheimer's disease are associated with lipid and cholesterol transport, suggesting defects in triglyceride usage that may underlie susceptibility to Alzheimer's disease. (4) Ingestion of MCT will lead to increased levels of blood ketone bodies and thereby provide energy to brain neurons. Hence, providing Alzheimer's disease and/or mild cognitive impairment patients with MCT will restore neuronal metabolism. Additionally, defects in neuronal metabolism in Huntington's Disease, Parkinson's Disease, and epilepsy and other related neurodegenerative diseases such as Wernicke-Korsakoff Disease and possibly schizophrenia will be benefited by high blood ketone levels, derived from MCT, that provide an energy source for brain cells.

It is also the novel insight of this invention that a combination of MCTs and therapeutic agents that increase the utilization of fatty acids by any mechanism are useful as a treatment and preventative measure for AD patients.

In one embodiment, the present invention provides a composition for the treatment of or prevention of Alzheimer's disease or mild cognitive impairment. This composition includes at least one compound capable of elevating ketone body concentrations in an amount effective for the treatment of or prevention of loss of cognitive function caused by reduced neuronal metabolism in dementia of Alzheimer's type or mild cognitive impairment. This composition also includes a therapeutic agent. The therapeutic agent includes at least one of the following: an anti-Alzheimer's agent, an anti-diabetic agent, an agent capable of increasing utilization of lipids, an anti-atherosclerotic agent, an anti-hypertensive agent, an anti-inflammatory agent, an anti-obesity agent, and combinations thereof. For convenience, when “anti-Alzheimer's agent” or any other therapeutic agent is referenced herein, such reference does not mean to refer to compounds capable of elevating ketone body concentrations, as defined herein. However, it is acknowledged that compounds of the invention, which are capable of elevating ketone body concentrations, are demonstrated to be potent anti-Alzheimer's agents.

In another embodiment, the present invention provides a method for treating dementia of Alzheimer's type or mild cognitive impairment. The method includes the steps of identifying a mammal having, or at risk of dementia of Alzheimer's type or mild cognitive impairment. The method also includes administering to the mammal a first composition comprising at least one compound capable of elevating ketone body concentrations in an amount effective for the treatment of or prevention of loss of cognitive function caused by reduced neuronal metabolism in dementia of Alzheimer's type or mild cognitive impairment. The method also includes administering to the mammal a second composition comprising a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof.

In all embodiments, the invention provides compositions having at least one compound that is capable of elevating ketone body concentrations. Such compositions may also be referred to as a “first composition”. Such compounds are also collectively referred to as ketone body precursor compounds or ketogenic compounds. Such compounds include compounds such as, for example, MCT, MCFA, and prodrugs, metabolic precursors, etc., of ketone bodies. For example, in one embodiment, the compound capable of elevating ketone body concentrations in the body include one or more prodrugs, which can be metabolically converted to ketone bodies by the recipient host. As used herein, a prodrug is a compound that exhibits pharmacological activity after going through a chemical transformation in the body. A prodrug can also be referred to as a metabolic precursor if the conversion of the prodrug directly results in the formation of a ketone body. MCT and MCFA must first be oxidized to acetyl-CoA, then undergo several steps before being synthesized into ketone bodies. The class of ketone body precursor compounds include the compounds described hereinbelow. The ketone body precursor compounds, in one embodiment, are administered in a dosage required to increase blood ketone bodies to a level required to treat and/or prevent the occurrence of Alzheimer's disease, mild cognitive impairment, or other disease of reduced neuronal metabolism. Appropriate dosages of all of these compounds can be determined by one of skill in the art, particularly in view of the specific guidance provided for MCT.

A wide variety of prodrug formulations are known in the art. For example, prodrug bonds may be hydrolyzable or enzymatically degradable, such as esters or anhydrides, and amides.

Ketone body precursor compounds appropriate for the inventive compositions include any compounds that are capable of directly elevating ketone body concentrations in the body of a mammal, e.g., a patient, and may be determined by one of skill in the art. The ketone body precursor compound will be administered in a dosage required to increase blood ketone bodies to a level required to treat and prevent the occurrence of Alzheimer's disease, mild cognitive impairment, and other diseases of reduced neuronal metabolism. Ketone bodies are produced continuously by oxidation of fatty acids in tissues that are capable of such oxidation. The major organ for fatty acid oxidation is the liver. Under normal physiological conditions ketone bodies are rapidly utilized and cleared from the blood. Under some conditions, such as starvation or low carbohydrate diet, ketone bodies are produced in excess and accumulate in the blood stream. Compounds that mimic the effect of increasing oxidation of fatty acids will raise ketone body concentration to a level to provide an alternative energy source for neuronal cells with compromised metabolism. Since the efficacy of such compounds derives from their ability to increase fatty acid utilization and raise blood ketone body concentration, they are dependent on the embodiments of the present invention.

These compounds can mimic the effect of increasing oxidation of fatty acids and include but are not limited to ketone bodies, D-β-hydroxybutyrate and acetoacetate, and metabolic precursors of these. The term metabolic precursor, used in this embodiment, can refer to compounds that comprise 1,3 butane diol, acetoacetyl or D-β-hydroxybutyrate moieties such as acetoacetyl-1-1,3 butanediol, acetoacetyl-D-β-hydroxybutyrate, and acetoacetylglycerol. Esters of any such compound with monohydric, dihydric, or trihydric alcohols are also included in yet another embodiment. Metabolic precursors also include polyesters of D-β-hydroxybutyrate, and acetoacetate esters of D-β-hydroxybutyrate. Polyesters of D-β-hydroxybutyrate include oligomers of this polymer designed to be readily digestible and/or metabolized by humans or mammals. These preferably are of 2 to 100 repeats long, typically 2 to 20 repeats long, and most conveniently from 3 to 10 repeats long. The preparation and use of such metabolic precursors is detailed in Veech, WO 98/41201, and Veech, WO 00/15216, each of which is incorporated by reference herein in its entirety. Examples of poly D-β-hydroxybutyrate or terminally oxidized poly-D-β-hydroxybutyrate esters usable as ketone body precursors are given below:

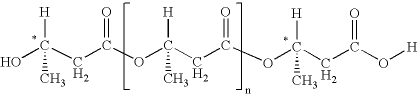

wherein n are integers of 0 to 1,000; another compound is

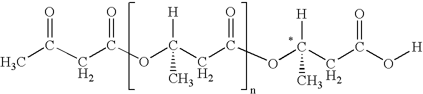

wherein n are integers of 1 to 1,000; another compound is

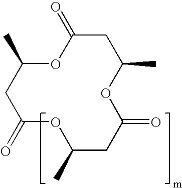

In each case, n is selected such that the polymer or oligomer is readily metabolized on administration to a human or mammal body to provide elevated ketone body levels in blood. Values of n are integers of 0 to 1,000, more preferably 1 to 200, still more preferably 1 to 50, most preferably 1 to 20, particularly conveniently from 3 to 5. M is an integer of 1 or more, a complex thereof with one or more cations or a salt thereof for use in therapy or nutrition. Examples of cations and typically physiological salts are described herein, and additionally include sodium, potassium, magnesium, and calcium, each balanced by a physiological counter-ion forming a salt complex. Examples are L-lysine, L-arginine, methyl glucamine, and others known to those skilled in the art.

Also included in the definition of a ketone body precursor are several other ketone body precursor compounds useful for treating diseases of reduced neuronal metabolism, such as Alzheimer's disease and mild cognitive impairment, including esters of polyhydric alcohols, 3-hydroxyacid esters and glycerol esters, as described more fully hereinbelow. As used herein, “derivative” refers to a compound or portion of a compound that is derived from is theoretically derivable from a parent compound; the term “hydroxyl group” is represented by the formula —OH; the term “alkoxy group” is represented by the formula —OR, where R can be an alkyl group, including a lower alkyl group, optionally substituted with an alkenyl, alkynyl, aryl, aralkyl, cycloalkyl, halogenated alkyl, or heterocycloalkyl group, as defined below; the term “ester” is represented by the formula —OC(O)R, where R can be an alkyl, alkenyl, alkynyl, aryl, aralkyl, cycloalkyl, halogenated alkyl, or heterocycloalkyl group, as defined below; the term “alkyl group” is defined as a branched or unbranched saturated hydrocarbon group of 1 to 24 atoms, such as methyl, ethyl, n-propyl, isopropyl, n-butyl, isobuyl, t-butyl, pentyl, hexyl, heptyl, octyl, decyl, dodecyl, tetradecyl, hexadecyl, eicosyl, tetracosyl and the like. A “lower alkyl” group is a saturated branched or unbranched hydrocarbon having from 1 to 10 carbon atoms; the term “alkenyl group” is defined as a hydrocarbon group of 2 to 24 carbon atoms and structural formula containing at least one carbon-carbon double bond; the term “alkynyl group” is defined as a hydrocarbon group of 2 to 24 carbon atoms and structural formula containing at least one carbon-carbon double bond; the term “alkynyl group” is defined as a hydrocarbon group of 2 to 24 carbon atoms and structural formula containing at least one carbon-carbon triple bond; the term “halogenated alkyl group” is defined as an alkyl group as defined above with one or more hydrogen atoms present on these groups substituted with a halogen (F, Cl, Br, I); the term “cycloalkyl group” is defined as a non-aromatic carbon-based ring composed of at least three carbon atoms. Examples of cyclalkyl groups include, but are not limited to, cyclopropyl, cyclobutyl, cyclopentyl, cyclohexyl, etc. The term “heterocycloalkyl group: is a cycloalkyl group as defined above where at least one of the carbone atoms of the ring is substituted with a heteroatom such as, but not limited to, nitrogen, oxygen, sulfur, or phosphorous; the term “aliphatic group” is defined as including alkyl, alkenyl alkynyl, or halogenated alkyl and cycloalkyl groups as defined above. A “lower aliphatic group” is an aliphatic group that contains from 1 to 10 carbon atoms; the term “aryl group” is defined as any carbon-based aromatic group including, but not limited to, benzene, napthalene, etc. The term “aromatic” also includes “heteroaryl group” which is defined as an aromatic group that has at least one heteroatom incorporated with the ring of the aromatic group. Examples of heteroatoms include, but are not limited to nitrogen, oxygen, sulfur, and phosphorous. The aryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted; the term “aralkyl” is defined as an aryl group having an alkyl group, as defined above, attached to the aryl group. An example of an aralkyl group is a benzyl group; “esterification” refers to the reaction of an alcohol with a carboxylic acid or a carboxylic acid derivative to give an ester; “transesterification” refers to the reaction of an ester with an alcohol to form a new ester compound. The term “3-hydroxybutyrate” is used interchangeably with the term “3-hydroxybutyric acid.”

In one embodiment, a compound capable of elevating ketone body concentrations includes esters of polyhydric alcohols of the formula

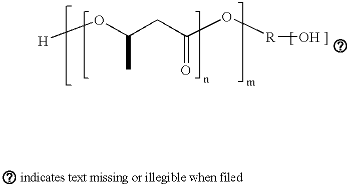

wherein R is a polyhydric alcohol residue; n, m and x represent integers; and m is less than or equal to x.

Physiologically compatible alcohols suitable for forming esters with (R)-3-hydroxybutyrate and derivatives thereof include monohydric and polyhydric alcohols. Esters of polyhydric alcohols delivery a higher density of (R)-3-hydroxybutyrate equivalents per equivalent of (R)-3-hydroxybutyrate derivative using shorter (R)-3-hydroxybutyrate oligomers. Shorter oligomers generally are more readily hydrolyzed to give elevated concentrations of (R)-3-hydroxybutyrate in blood. Examples of polyhyric alcohols suitable for preparing such esters include carbohydrates including, without limitation, altrose, arabinose, dextrose, erythrose, fructose, galactose, glucose, gulose, idose, lactose, lyxose, mannose, ribose, sucrose, talose, threose, xylose and the like. Additional examples of carbohydrates useful for preparing (R)-3-hydroxybutyrate derivatives include amino derivatives, such as galactosamine, glucosamine and mannosamine, including N-acetyl derivatives, such as N-acetylglucosamine and the like. Examples of carbohydrates also include carbohydrate derivatives, such as alkyl glycosides. Examples of carbohydrates, also include, without limitation, glycerol, mannitol, ribitol, sorbitol, threitol, xylitol, and the like. The enantiomers of the above-listed carbohydrates and carbohydrate alcohols can also be used to prepare (R)-3-hydroxybutyrate derivatives according to the above formula.

Embodiments include compounds where n is from 1 to about 100; wherein X is from 1 to about 20, wherein m is from 1 to about 20. One embodiment includes a compound wherein R is (R)-1,3-butane diol.

In another embodiment, compounds capable of elevating ketone body concentrations include compounds of the formula

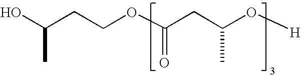

and also

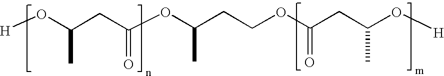

where n and m independently are integers from 1 to about 100. In some embodiments, n and m are the same; n and m are different; and wherein n and m are 3.

In addition, compounds capable of elevating ketone body concentrations include ester compounds of R-3-hydroxybutyrate according to the formula

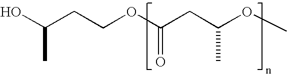

wherein n is an integer from 1 to about 100. In one embodiment, n is 3.

Other compounds capable of elevating ketone body levels include 3-hydroxyacids. The compositions include 3-hydroxyacids, linear or cyclic oligomers thereof, esters of the 3-hydroxyacids or oligomers, derivatives of 3-hydroxyacids, and combinations thereof. In one embodiment, the compositions include the cyclic macrolide of R-3-hydroxyacids containing 3, 4, or 5 monomeric subunits. 3-hydroxyacids include 3-hydroxybutyric acid, 3-hydroxyvaleric acid, 3-hydroxyhexanoic acid and 3-hydroxyheptanoic acid. In some embodiments, the length of the oligomer must be such that the derivative has a suitable digestion rate for sustained release of monomer. In another embodiment, the cyclic trimer (triolide) is used in a combination with other cyclic oligolides or linear esters and/or mixtures of both.

In another embodiment, the present invention includes, as a compound capable of raising ketone body levels, 3-hydroxyacids of the formula:

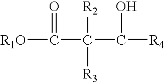

wherein, R1 is selected from hydrogen, methyl, alkyl, alkenyl, aryl, arylalkyl, heteroalkyl, heteroaryl, thiol, disulfide, ether, thiol ether, amine, amide, and halogen. R2 and R3 are independently selected from hydrogen, methyl, alkyl, alkenyl, aryl, arylalkyl, heteroalkyl, heteroaryl, thiol, disulfide, ether, thiol ether, amine, amide, halogen, hydroxy, ester, nitrogen-substituted radicals, and/or oxygen-substituted radicals. R4 is selected from hydrogen, alkyl, alkenyl, aryl, arylalkyl, heteroalkyl, heteroaryl, thiol, disulfide, ether, thiol ether, amine, amide, halogen, hydroxy, ester, nitrogen-substituted radicals, and/or oxygen substituted radicals. Further, when R4 is not hydrogen or a halogen, R3 can be a direct bond to R4 and R4 can be methyl.

In another embodiment of the present invention, another compound capable of elevating ketone body concentrations includes glycerol esters of the formula

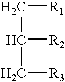

wherein two or three of the groups R1, R2 and R3 independently of each other, are one or more of the groups acetoacetate, alpha-ketopropionate, beta-hydroxybutyrate and alpha-hydroxypropionate, and when only two of the groups R1, R2 and R3 are any of said groups, the third of them is a hydroxy group or a residue of a saturated or unsaturated fatty acid containing 2 to 24 carbon atoms. Other glycerol esters are envisioned, particularly the not readily water soluble glycerides of at least one keto or hydroxy acid, having the formula:

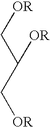

wherein one R group is hydrogen, and two R groups are (—COCH₂, —COCH₃). Additionally, wherein each R is the same or different and is hydrogen, or (—COCH₂, —COCH₃), provided that at least one R is not hydrogen and wherein R′ is a linear acid ester of even carbon number from 2 to 20 carbons.

In one embodiment, a glycerol ester includes medium chain triglycerides (MCT) referring to any glycerol molecule ester-linked to three fatty acid molecules, each fatty acids having a carbon chain of 5-12 carbons. The structured lipids of the invention may be prepared by any process known in the art, such as, direct esterification, rearrangement, fractionation, transesterification, and the like. For example, the lipids may be prepared by the rearrangement of a vegetable oil such as coconut oil. For example, MCT containing 1-10% C6, 30-60% C8, 30-60% C10, and 1-10% C12 are commonly derived from palm and coconut oils. MCT containing greater than about 95% C8 at R1, R2, and R3 can be made by semi-synthetic esterification of octanoic acid to glycerin. Such MCT behave similarly and are encompassed with the term MCT as used herein.

In one embodiment, the method comprises the use of MCTs wherein R1 is a fatty acid containing a six-carbon backbone (tri-C6:0). Tri-C6:0 MCT are absorbed very rapidly by the gastrointestinal tract in a number of animal model systems (Odle 1997). The high rate of absorption results in rapid perfusion of the liver, and a potent ketogenic response. In another preferred embodiment, the method comprises the use of MCTs wherein R1 is a fatty acid containing an eight-carbon backbone (tri-C8:0). Additionally, utilization of tri-C6:0 MCT and tri-C8:0 MCT can be increased by emulsification. Emulsification of lipids increases the surface area for action by lipases, resulting in more rapid hydrolysis. Methods for emulsification of these triglycerides are well known to those skilled in the art.

The inventor has demonstrated the efficacy of tri-C8:0 medium chain triglycerides (MCT) in the treatment of AD. In the Examples section, the inventor reveals several key aspects of the invention. First, MCT induce elevated ketone body levels in the elderly. Second, MCT induce different ketone body levels in different subjects based on their apolipoprotein E genotype. Third, MCT treatments result in improved performance on established Alzheimer Disease cognitive measures (ADAS-cog). Fourth, improved performance on ADAS-cog test was influenced by the subject's apolipoprotein E genotype. Fifth, higher ketone body levels were associated with greater improvement in a second measure of cognitive performance (the paragraph recall test) with MCT treatment.

In one embodiment, the compound capable of elevating ketone body concentrations comprises medium chain triglycerides (MCT) of the formula:

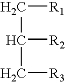

wherein the R1, R2, and R3 esterified to the glycerol backbone are each independently fatty acids having 5-12 carbon chains. In another embodiment, the compound comprises MCT wherein R1, R2, and R3 are fatty acids containing an eight-carbon backbone (tri-C8:0). In another embodiment, the compound comprises MCT wherein R1, R2, and R3 are fatty acids containing a ten-carbon backbone (tri-C10:0). In another embodiment, the compound comprises MCT wherein R1,R2, and R3 are a mixture of C8:0 and C10:0 fatty acids. In another embodiment, the compound comprises MCT wherein R1, R2 and R3 are a mixture of C6:0, C8:0, C10:0, and C12:0 fatty acids. In one embodiment, greater than 95% of the R1, R2, and R3 carbon chains are 8 carbons in length, and the remaining R1, R2, and R3 carbon chains are 6-carbon or 10-carbon chains. In another embodiment, about 50% of the R1, R2, and R3 carbon chains are 8 carbons in length and about 50% of the R1, R2 and R3 carbon chains are about 10 carbons in length. In another embodiment, greater than 95% of R1, R2 and R3 carbon chains of the MCT are 8 carbons in length. In yet another embodiment, the R1, R2, and R3 carbon chains are 6-carbon or 10-carbon chains. The composition of the MCT does not appear to have a discernable difference as to use or effect since MCT with >95% C8 and MCT with 45% C8-55% C10 have been used, and other studies have used MCTs with other compositions. Additionally, utilization of MCT can be increased by emulsification. Emulsification of lipids increases the surface area for action by lipases, resulting in more rapid hydrolysis and release of MCFA. Methods for emulsification of these triglycerides are well known to those skilled in the art.

In another embodiment, the composition or first composition comprises NEOBEE 895 (Stepan, Inc.), comprising triglycerides, wherein approximately 97% of the R1, R2, and R3 carbon chains are 8 carbons in length and the triglcyeride has a specific gravity (at 25° C.) of 0.958, so 1 mL equals 0.958 gm of MCT. In practice, the inventors have used multiple different MCT compositions, for example, NEOBEE 895 and NEOBEE 1053, and no discernable difference in response or in ketone body formation attributable to the exact composition of MCT have been evidenced in the inventors' studies.

Generally, an effective amount is an amount effective to either (1) reduce the symptoms of the disease sought to be treated or (2) induce a pharmacological change relevant to treating the disease sought to be treated. For Alzheimer's Disease, an effective amount includes an amount effective to: increase cognitive scores; slow the progression of dementia; or increase the life expectancy of the affected patient. Effective amount also refers to an amount of compound or composition as described herein that is effective to achieve a particular biological result. In various embodiments, effective amount refers to an amount suitable to reverse, reduce, prevent, or delay a decline in Alzheimer's disease, mild cognitive impairment. Effectiveness for treatment of the aforementioned conditions may be assessed by improved results for at least one neuropsychological test, and includes any neuropsychological tests known in the art for assessing Alzheimer's disease, mild cognitive impairment, or other disease of reduced neuronal metabolism. Examples of such neuropsychological tests include ADAS-cog, MMSE, Stroop Color Word Interference Task, Logical Memory subtest of the Wechsler Memory Scale-III, Clinician's Dementia Rating, and Clinician's Interview Based Impression of Change. Effectiveness for treatment of the aforementioned conditions include improvements in the proper physiological activity of the brain, such as mental stability, memory/recall abilities, problem solving abilities, reasoning abilities, thinking abilities, judging abilities, capacity for learning, perception, intuition, awareness, attention, as measured by any means suitable in the art.

Decline of any of the foregoing categories or specific types of qualities or functions in an individual is generally the opposite of an improvement or enhancement in the quality or function. An “effective amount” (as discussed above) of a composition of the invention may be an amount required to prevent decline, to reduce the extent or rate of decline, or delay the onset or progression of a decline, or lead to an improvement from a previous decline. Prevention, reduction, or delay of a decline can be considered relative to a cohort that does not receive the treatment. Prevention, reduction or delay of a decline may also be measured and considered on an individual basis, or in some embodiments, on a population basis.

Compounds that are referred to as “anti-“X” agents” comprise agents that (1) reduce the symptoms of the disease sought to be treated or (2) induce a pharmacological change relevant to treating the disease sought to be treated.

In another embodiment, the invention provides a method of treating or preventing dementia of Alzheimer's type, mild cognitive impairment, or other loss of cognitive function caused by reduced neuronal metabolism, comprising administering a first composition comprising an effective amount of free fatty acids, which may be derived from medium chain triglycerides, to a patient in need thereof. Because MCT are metabolized to produce medium chain fatty acids, which are oxidized, the administration of free fatty acids and/or ketone bodies have the same effect as the administration of MCT themselves. The method further provides administering a second composition comprising an anti-Alzheimer's agent, an anti-diabetic agent, an agent capable of increasing utilization of lipids, an anti-atherosclerotic agent, an anti-hypertensive agent, an anti-inflammatory agent, an anti-obesity agent, and/or combinations thereof.

Therapeutically effective amounts of the compositions of the invention, including the first composition and the second composition, can be any amount or dose sufficient to bring about the desired anti-dementia effect and depend, in part, on the severity and stage of the condition, the size and condition of the patient, as well as other factors readily known to those skilled in the art. The dosages can be given as a single dose, or as multiple doses, for example, provided over the course of several weeks.

In one embodiment, the compounds capable of elevating ketone body levels, MCT or fatty acids are administered orally. In another embodiment, the compounds are administered intravenously. Oral administration of compounds such as MCT and preparations of intravenous compositions such as MCT solutions are well known to those skilled in the art.

Oral and intravenous administration of MCT or fatty acids result in hyperketonemia. Hyperketonemia results in ketone bodies being utilized for energy in the brain even in the presence of glucose. Additionally, hyperketonemia results in a substantial (39%) increase in cerebral blood flow (Hasselbalch et al. 1996). Hyperketonemia has been reported to reduce cognitive dysfunction associated with systemic hypoglycemia in normal humans (Veneman et al. 1994). Please note that systemic hypoglycemia is distinct from the local defects in glucose metabolism that occur in AD.

Administration of the compositions, including the first composition and/or the second composition, can be on an as-needed or as-desired basis. Where the composition is the first composition, for example, the composition can be administered once monthly, once weekly, daily, or more than once daily. Similarly, administration can be every other day, week, or month, every third day, week, or month, every fourth day, week, or month, and the like. Administration can be multiple times per day. When utilized as a supplement to ordinary dietary requirements, the composition may be administered directly to the mammal or otherwise contacted with or admixed with daily food or beverage. When utilized as a daily food or beverage, administration techniques will be known to those of skill in the art. Administration can also be carried out on a regular basis, for example, as part of a treatment regimen in the mammal. A treatment regimen may comprise causing the regular ingestion by the mammal of an inventive composition or inventive first and second compositions in an amount effective to enhance characteristics as defined above. Regular ingestion can be once a day, or two, three, four, or more times per day, on a daily or weekly basis. Similarly, regular administration can be every other day or week, every third day or week, every fourth day or week, every fifth day or week, every sixth day or week, and in such a regimen, administration can be multiple times per day. The goal of regular administration is to provide the mammal with optimal dose of an inventive compositions, as exemplified herein.

The compositions provided herein, are, in one embodiment, intended for “long term” consumption, sometimes referred to herein as for “extended” periods. Long-term administration as used herein generally refers to periods in excess of one month. Periods of longer than two, three, or four months comprise one embodiment of the instant invention. Also included are embodiments comprising more extended periods that include longer than 5, 6, 7, 8, 9, or 10 months. Periods in excess of 11 months or one year are also included. Longer-term use extending over 1, 2, 3, 4, 5, 6, 7, 8, 10, 12, 14, 16, 18, 20 or more years is also contemplated. In some cases, it is envisioned that the patient would continue consuming the compositions for the remainder of its life, on a regular basis as discussed hereinabove. Regular basis as used herein refers to at least weekly dosing with or consumption of the compositions. More frequent dosing or consumption, such as twice or thrice weekly are also included. Also included are regimens that include at least once daily consumption. The skilled artisan will appreciate that the blood (urine or cerebral spinal fluid) levels of ketone bodies, or a specific ketone body, achieved may be a valuable measure for determining dosing frequency. Any frequency, regardless of whether expressly exemplified herein, that allows maintenance of a blood level of the measured compound within acceptable ranges can be considered useful herein. The skilled artisan will appreciate that dosing frequency will be a function of the composition that is being consumed or administered, and some compositions may require more or less frequent administration to maintain a desired blood level of the measured compound (e.g., a ketone body).

This invention also provides a compound capable of elevating ketone body levels for the treatment or prevention of dementia of Alzheimer's type, or other loss of cognitive function caused by reduced neuronal metabolism, comprising medium chain triglycerides. In a preferred embodiment, the ketogenic compound is provided in administratively convenient formulations of the compositions including dosage units incorporated into a variety of containers. Dosages of the ketogenic compound, such as MCT, are preferably administered in an effective amount, in order to produce ketone body concentrations sufficient to increase the cognitive ability of patients afflicted with AD or other states of reduced neuronal metabolism, as discussed hereinabove.

In one embodiment, the ketogenic compounds are administered orally. In another embodiment, the ketogenic compounds are administered intravenously. Oral administration of MCT and other keotgenic compound preparations of intravenous MCT and/or other ketogenic solutions are known in the art.

In one embodiment, the composition (as discussed hereinabove, composition may refer to either the first, second or other composition referenced herein) increases the circulating concentration of at least one type of ketone body in the mammal or patient. In one embodiment, the circulating ketone body is D-β-hydroxybutyrate. The amount of circulating ketone body can be measured at a number of times post administration, and in one embodiment, is measured at a time predicted to be near the peak concentration in the blood, but circulating ketone body can also measured before or after the predicted peak blood concentration level. Measured amounts at these off-peak times are then optionally adjusted to reflect the predicted level at the predicted peak time. In one embodiment, the predicted peak time is at about two hours. Peak circulating blood level and timing can vary depending on factors known to those of skill in the art, including individual digestive rates, co-ingestion or pre- or post-ingestion of foods, beverages, and so on, as known to those of skill in the art. In one embodiment, the peak blood level reached of D-β-hydroxybutyrate is between about 0.05 millimolar (mM) to about 50 mM. Another way to determine whether blood levels of D-β-hydroxybutyrate are raised to about 0.05 to about 50 mM in the blood is to determine D-β-hydroxybutyrate urinary excretion, where a level which corresponds to the foregoing blood levels is in a range of about 5 milligrams per deciliter (mg/dL) to about 160 mg/dL. In other embodiments, the peak blood level D-β-hydroxybutyrate is raised to about 0.15 to about 2 mM, to about 0.15 to about 0.3 mM. In other embodiments, the peak blood level of D-β-hydroxybutyrate is raised to at least about 0.05 mM, to at least about 0.1 mM, to at least about 0.15 mM, to at least about 0.2 mM, to at least about 0.5 mM, to at least about 1 mM, to at least about 2 mM, to at least about 2.5 mM, to at least about 3 mM, to at least about 4 mM, to at least about 5 mM, to at least about 10 mM, to at least about 20 mM, to at least about 30 mM, to at least about 40 mM, to at least about 50 mM. In another embodiment, the circulating concentration of at least one type of ketone body are levels of about 0.1 mM; in the range of 0.1 to 50 mM, in the range of 0.2-20 mM, in the range of 0.3-5 mM, and in the range of 0.5-2 mM.

Effective amounts of dosages of compounds for the inventive compositions, i.e., compounds capable of elevating ketone body concentrations in an effective amount, in some embodiments, the first composition, will be apparent to those skilled in the art, and can be conveniently determined by determining the amount of ketone body generated in the blood. Where the compound capable of elevating ketone body levels is MCT, the MCT dose, in one embodiment, will be in the range of 0.05 g/kg/day to 10 g/kg/day of MCT. More preferably, the dose will be in the range of 0.25 g/kg/day to 5 g/kg/day of MCT. More preferably, the dose will be in the range of 0.5 g/kg/day to 2 g/kg/day of MCT. In other embodiments, the dose will be in a range of about 0.1 g/kg/day to about 2 g/kg/day. In other embodiments, the dose of MCT is at least about 0.05 g/kg/day, at least about 0.1 g/kg/day, at least about 0.15 g/kg/day, at least about 0.2 g/kg/day, at least about 0.5 g/kg/day, at least about 1 g/kg/day, at least about 1.5 g/kg/day, at least about 2 g/kg/day, at least about 2.5 g/kg/day, at least about 3 g/kg/day, at least about 4 g/kg/day, at least about 5 g/kg/day, at least about 10 g/kg/day, at least about 15 g/kg/day, at least about 20 g/kg/day, at least about 30 g/kg/day, at least about 40 g/kg/day, and at least about 50 g/kg/day.

Convenient unit dosage containers and/or formulations include tablets, capsules, lozenges, troches, hard candies, nutritional bars, nutritional drinks, metered sprays, creams, and suppositories, among others. The compositions may be combined with a pharmaceutically acceptable excipient such as gelatin, oil, and/or other pharmaceutically active agent(s). For example, the compositions may be advantageously combined and/or used in combination with other therapeutic or prophylactic agents, different from the subject compounds. In many instances, administration in conjunction with the subject compositions enhances the efficacy of such agents. For example, the compounds may be advantageously used in conjunction with antioxidants, compounds that enhance the efficiency of glucose utilization, and mixtures thereof, (see e.g. Goodman et al. 1996).

In a preferred embodiment the human subject is intravenously infused with MCT, MCFA (medium chain fatty acids) and/or ketone bodies directly, to a level required to treat and prevent the occurrence of Alzheimer's Disease. Preparation of intravenous lipid, and ketone body solutions is well known to those skilled in the art.

Ketone bodies are used by neurons as a source of Acetyl-CoA. Acetyl-CoA is combined with oxaloacetate to form citrate in the Krebs' cycle, or citric acid cycle (TCA cycle). It is important for neurons to have a source of Acetyl-CoA as well as TCA cycle intermediates to maintain efficient energy metabolism. Yet, neurons lose TCA cycle intermediates to synthesis reactions, such as the formation of glutamate. Neurons also lack pyruvate carboxylase and malic enzyme so they cannot replenish TCA cycle intermediates from pyruvate (Hertz, Yu et al. 2000). Accordingly, the present invention discloses that a combination of ketone bodies with a source of TCA cycle intermediates will be beneficial to conditions of reduced neuronal metabolism. TCA cycle intermediates are selected from a group consisting of citric acid, aconitic acid, isocitric acid, α-ketoglutaric acid, succinic acid, fumaric acid, malic acid, oxaloacetic acid, and mixtures thereof. One embodiment of the invention is a combination of TCA cycle intermediates with MCT in a formulation to increase efficiency of the TCA.

Another source of TCA cycle intermediates are compounds that are converted to TCA cycle intermediates within the body (TCA intermediate precursors). Examples of such compounds are 2-keto-4-hydroxypropanol, 2,4-dihydroxybutanol, 2-keto-4-hydroxybutanol, 2,4-dihydroxybutyric acid, 2-keto-4-hydroxybutyric acid, aspartates as well as mono- and di-alkyl oxaloacetates, pyruvate and glucose-6-phosphate. Accordingly, the present invention discloses that a combination of TCA intermediate precursors with ketone bodies will be beneficial for the treatment and prevention of diseases resulting from reduced metabolism. In addition, the present invention discloses that MCT combined with TCA intermediate precursors will be beneficial for the treatment and prevention of diseases resulting from reduced metabolism.

The present invention further discloses that additional sources of TCA cycle intermediates and Acetyl-CoA can be advantageously combined with ketone body therapy. Sources of TCA cycle intermediates and Acetyl-CoA include mono- and di-saccharides as well as triglycerides of various chain lengths and structures.

Further benefit can be derived from formulation of a pharmaceutical composition, including a first composition and/or second composition, that includes metabolic adjuvants. Metabolic adjuvants include vitamins, minerals, antioxidants and other related compounds. Such compounds may be chosen from a list that includes but is not limited to; ascorbic acid, biotin, calcitriol, cobalamin, folic acid, niacin, pantothenic acid, pyridoxine, retinol, retinal (retinaldehyde), retinoic acid, riboflavin, thiamin, α-tocopherol, phytylmenaquinone, multiprenylmenaquinone, calcium, magnesium, sodium, aluminum, zinc, potassium, chromium, vanadium, selenium, phosphorous, manganese, iron, fluorine, copper, cobalt, molybdenum, iodine. Accordingly a combination of ingredients chosen from: metabolic adjuvants, compounds that increase ketone body levels, and TCA cycle intermediates, will prove beneficial for treatment and prevention of diseases associated with decreased metabolism, including Alzheimer's disease, Parkinson's Disease, Huntington's Disease, and epilepsy.

With regard to epilepsy, the prior art provides descriptions of ketogenic diets in which fat is high and carbohydrates are limited. In summary, the rationale of such diets is that intake of high amounts of fat, whether long-chain or medium-chain triglycerides, can increase blood ketone levels in the context of a highly regimented diet in which carbohydrate levels are absent or limited. Limitation of carbohydrate and insulin are believed to prevent re-esterification in adipose tissue. In contrast to the prior art, the present invention provides for and claims the administration of medium chain triglycerides outside of the context of the ketogenic diet. Furthermore, the EXAMPLES section below provides exemplary formulations which include carbohydrates.

Although the ketogenic diet has been known for decades, there does not appear to be any prior art teaching or suggesting that MCT therapy be used to treat Alzheimer's disease or other cognitive disorders.

Additional metabolic adjuvants include energy enhancing compounds, such as Coenzyme CoQ-10, creatine, L-carnitine, n-acetyl-carnitine, L-carnitine derivatives, and mixtures thereof. These compounds enhance energy production by a variety of means. Carnitine will increase the metabolism of fatty acids. CoQ10 serves as an electron carrier during electron transport within the mitochondria. Accordingly, addition of such compounds with MCT will increase metabolic efficiency especially in individuals who may be nutritionally deprived.

Administration of MCT, and especially triglycerides composed of C6 and C8 fatty acid residues, result in elevated ketone body levels even if large amounts of carbohydrate are consumed at the same time (for overview see (Odle 1997); see also U.S. Patent Provisional Patent Application Ser. No. 60/323,995, “Drug Targets for Alzheimer's Disease and Other Diseases Associated with Decreased Neuronal Metabolism,” filed Sep. 21, 2001). The advantages of the Applicant's approach are clear, since careful monitoring of what is eaten is not required and compliance is much simpler.

In one embodiment, the invention comprises the co administration of emulsified tri-C6:0 MCT and L-carnitine or a derivative of L-carnitine. Slight increases in MCFA oxidation have been noted when MCT are combined with L-carnitine (Odle, 1997). Thus in the present invention emulsified MCT are combined with L-carnitine at doses required to increase the utilization of said MCT. The dosage of L-carnitine and MCT will vary according to the condition of the host, method of delivery, and other factors known to those skilled in the art, and will be of sufficient quantity to raise blood ketone levels to a degree required to treat and prevent Alzheimer's Disease. Derivatives of L-carnitine which may be used in the present invention include but are not limited to decanoylcarnitine, hexanoylcarnitine, caproylcarnitine, lauroylcarnitine, octanoylcarnitine, stearoylcarnitine, myristoylcarnitine, acetyl-L-carnitine, O-Acetyl-L-carnitine, and palmitoyl-L-carnitine. In one embodiment, the invention provides a formulation comprising a mixture of MCT and carnitine to provide elevated blood ketone levels. The nature of such formulations will depend on the duration and route of administration. Such formulations will be in the range of 0.05 g/kg/day to 10 g/kg/day of MCT and 0.05 mg/kg/day to 10 mg/kg/day of carnitine or its derivatives. In one embodiment, an MCT dose will be in the range of 0.05 g/kg/day to 10 g/kg/day of MCT. More preferably, the dose will be in the range of 0.25 g/kg/day to 5 g/kg/day of MCT. More preferably, the dose will be in the range of 0.5 g/kg/day to 2 g/kg/day of MCT. In some embodiments, a carnitine or carnitine derivative dose will be in the range of 0.05 g/kg/day to 10 g/kg/day. More preferably, the carnitine or carnitine derivative dose will be in the range of 0.1 g/kg/day to 5 g/kg/day. More preferably, the carnitine or carnitine derivative dose will be in the range of 0.5 g/kg/day to 1 g/kg/day. Variations will necessarily occur depending on the formulation and/or host, for example.

A particularly preferred formulation comprises a range of 1-500 g of emulsified MCT combined with 1-2000 mg of carnitine. Amounts of MCT can be at least about 1 g, at least about 10 g, at least about 50 g, at least about 100 g, at least about 150 g, at least about 200 g, at least about 250 g, at least about 300 g, at least about 400 g. Amounts of carnitine can be at least about 1 mg, at least about 50 mg, at least about 100 mg, at least about 250 mg, at least about 500 mg, at least about 1000 mg, at least about 1250 mg, or at least about 1500 mg. An even more preferred formulation comprises 50 g MCT (95% triC8:0) emulsified with 50 g of mono- and di-glycerides combined with 500 mg of L-carnitine. Such a formulation is well tolerated and induces hyperketonemia for 3-4 hours in healthy human subjects.

Dosage amounts of MCT can also be measured in terms of grams of MCT per kg of body weight (BW) of the mammal. The daily dose of MCT can range from about 0.01 g/kg to about 10 g/kg BW of the mammal. Optionally, the daily dose of MCT is from about 0.1 g/kg to about 5 g/kg BW of the mammal. Optionally, the daily dose of MCT is from about 0.2 g/kg BW of the mammal to about 3 g/kg BW of the mammal. Optionally, the daily dose of MCT is from about 0.5 g/kg to about 2 g/kg of the mammal.

In some embodiments, the inventive compounds may be co-administered with a carbohydrate source or co-formulated with a carbohydrate source. A carbohydrate source can include more than one type of carbohydrate. Carbohydrates or saccharides are generally simple molecules that are straight-chain aldehydes or ketones with many hydroxyl groups added, usually one on each carbon atom that is not part of the aldehyde or ketone functional group. A carbohydrate may be a monosaccharide, a disaccharide, a polysaccharide and/or an oligosaccharide. Appropriate carbohydrates for the invention are carbohydrates, which are, upon digestion in a mammal, capable of yielding at least a portion of the carbohydrate as a monosaccharide. In one embodiment, the carbohydrate is a monosaccharide, and optionally is glucose, fructose and/or galactose. In another embodiment, the carbohydrate is a disaccharide, and optionally is sucrose and/or lactose.

In another embodiment, the invention further comprises determination of the patient's genotype or particular alleles. This method can further comprise selecting patients for treatment based on the results of the determination. In one embodiment, the patient's alleles for apolipoprotein E gene are determined. In some examples, the inventor teaches that non-E4 carriers performed better than those with the E4 allele when elevated ketone body levels were induced with MCT. In addition, those with the E4 allele had higher fasting ketone body levels and the levels continued to rise at the two-hour time interval. Therefore, E4 carriers may require higher ketone levels or agents that increase the ability to use the ketone bodies that are present. Accordingly, in one embodiment for those with the E4 allele dosages to administer include a dose of MCT combined with agents that increase the utilization of fats, MCT or ketone bodies. Examples of agents that increase utilization of fatty acids may be selected from a group comprising of, but not limited to, non-steroidal anti-inflammatory agents (NSAIDs), statin drugs (such as Lipitor® and Zocor®) and fibrates (as discussed elsewhere herein).

Key to the instant invention is the insight and experimental data that show that further benefit can be derived from formulation of a pharmaceutical composition comprising a compound capable of elevating ketone body concentrations in a patient, such as MCT, and an additional therapeutic agent, such as, for example, anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof. In one embodiment, the other therapeutic agents are ones used in the treatment of Alzheimer's Disease, Parkinson's Disease, Huntington's Disease, or epilepsy.

In some methods of the invention, both the ketogenic compound and the therapeutic agent(s), e.g., the first composition and the second composition, are administered to mammals (e.g., humans, male or female) using respective conventional methods. Administration of each composition referenced herein can be in a dosage form and schedule in accordance with current protocols, recommendations, or schedules known in the art for that composition and/or compound. In this embodiment, the administration of the ketogenic compound and the therapeutic agent(s) will be in accordance with protocols and/or dosing regimes specific to each, but will occur in a manner that administration of a ketogenic compound and a therapeutic agent(s) are at least partially overlapping in a specific mammal during a specific treatment regimen. In one embodiment, the administration of the ketogenic compound and the therapeutic agent(s) is substantially overlapping during a treatment regimen. In one embodiment, the treatment regimens for the first and second compositions will overlap sufficiently in order for the beneficial effects as noted herein to occur.

The ketogenic compound and the therapeutic agent(s) may also be employed together in the same oral dosage form or in separate oral dosage forms taken at the same time. The compositions described above may be administered in single or multiple doses of one to four times daily. It may be advisable to start a patient on a low dose combination and work up gradually to a high dose combination.

Tablets of various sizes can be prepared, e.g., of about 2 to 2000 mg in total weight, containing one or both of the active substances in the ranges described above, with the remainder being a physiologically acceptable carrier of other materials according to accepted pharmaceutical practice. These tablets can, of course, be scored to provide for fractional doses. Gelatin capsules can be similarly formulated. Liquid formulations can also be prepared by dissolving or suspending one or the combination of active substances in a conventional liquid vehicle acceptable for pharmaceutical administration.

Such therapeutic agents include cholinesterase inhibitors, acetylcholine synthesis modulators, acetylcholine storage modulators, acetylcholine release modulators, anti-inflammatory agents, estrogen or estrogen derivatives, insulin sensitizing agents, amyloid-β (Aβ) plaque removal agents (including vaccines), inhibitors of Aβ plaque formation, inhibitors of amyloid precursor protein (APP) processing enzymes, γ-secretase modulators, pyruvate dehydrogenase complex modulators, neurotrophic growth factors (e.g., BDNF, NGF), ceramides or ceramide analogs, and/or NMDA glutamate receptor antagonists for overview of such treatments see (Selkoe 2001; Bullock 2002)).

In another embodiment, the therapeutic agent is an anti-Alzheimer's agent and includes such agents as are known or found to be modulators of cholinesterase, acetylcholine synthesis modulators, acetylcholine storage modulators, acetylcholine release modulators, NMDA receptor antagonists, Aβ inhibitors, Aβ plaque removal agents (including vaccines), inhibitors of Aβ plaque formation, inhibitors of amyloid precursor protein processing enzymes, β-amyloid converting enzyme (BACE) inhibitors, β-secretase inhibitors, γ-secretase modulators, nerve growth factor agonists, hormone receptor blockade agents, neurotransmission modulators, anti-inflammatory agents, and combinations thereof. Preferred therapeutic agents include donepezil, rivastigmine, galantamine, and memantine.

In one embodiment, the anti-Alzheimer's agent is an inhibitor of cholinesterase. In one embodiment, the modulator of cholinesterase includes at least one of the following compounds: tacrine (Cognex), donepezil (Aricept), rivastigmine (Exelon) galantamine (Reminyl/Razadyne), physostigmine, neostigmine, Huperzine A, icopezil (CP-118954, 5,7-dihydro-3-[2-[1-(phenylmethyl)-4-piperidinyl]ethyl]-6H-pyrrolo-[4,5-f]-1,2-benzisoxazol-6-one maleate), ER-127528 (4-[(5,6-dimethoxy-2-fluoro-1-indanon)-2-yl]methyl-1-(3-fluorobenzyl)piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), Metrifonate (T-588; (−)—R-α-[[2-(dimethylamino)ethoxy]methyl]benzo[b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f]oxepine-10-methanamine), SDZ-220-581 ((S)-α-amino-5-(phosphonomethyl)-[1,1′-biphenyl]-3-propionic acid), and combinations thereof.

In one embodiment, the anti-Alzheimer's agent is an NMDA receptor antagonist. In one embodiment, the NMDA receptor antagonist includes memantine (Namenda/Exiba), neramexane (1,3,3,5,5-pentamethylcyclohexan-1-amine), and/or combinations thereof.

In another embodiment, the anti-Alzheimer's agent is an Aβ inhibitor, Aβ plaque removal agents (including vaccines), inhibitors of Aβ plaque formation, inhibitors of amyloid precursor protein processing enzymes, β-amyloid converting enzyme (BACE) inhibitors, β-secretase inhibitors, γ-secretase modulators. In another embodiment, the Aβ inhibitor is selected from the group consisting of tarenflurbil (Flurizan), tramiprosate (Alzhemed), clioquinol, PBT-2 (and other 8-hydroxyquinilone derivative described in US Patent Publication 2006/0089380), Aβ plaque removal agents (including vaccines), inhibitors of Aβ plaque formation, inhibitors of amyloid precursor protein processing enzymes, β-amyloid converting enzyme (BACE) inhibitors, β-secretase inhibitors, γ-secretase modulators (LY450139; N—[N-(3,5-difluorophenacetyl)-L-alanyl)-S-phenylglycine t-butyl ester), and combinations thereof.

In another embodiment, the anti-Alzheimer's agent is a nerve growth factor agonist. In another embodiment, the nerve growth factor agonist is xaliproden or brain derived neurotrophic factor (BDNF) or nerve growth factor (NGF). In another embodiment, the anti-Alzheimer's agent is a hormone receptor blockade agent. In another embodiment, the hormone receptor blockade agent is leuproelide or a derivative thereof. In another embodiment, the anti-Alzheimer's agent is a neurotransmission modulator. In another embodiment, the neurotransmission modulator is ispronicline.

In yet another embodiment, the anti-Alzheimer's agent is an anti-inflammatory agent. In another embodiment, the anti-inflammatory agent is selected from the group consisting of salicylates, aspirin, amoxiprin, benorilate, choline magnesium salicylate, diflunisal, faislamine, methyl salicylate, magnesium salicylate, salicyl salicylate, diclofenac, aceclofenac, acemetacin, bromfenac, etodolac, indometacin, nabumetone, sulindac, tolmetin, ibuprofen, carprofen, fenbufen, fenoprofen, flurbiprofen, ketoprofen, ketorolac, loxoprofen, naproxen, tiaprofenic acid, suprofen, mefenamic acid, meclofenamic acid, phenylbutazone, azapropazone, metamizole, oxyphenbutazone, sulfinprazone, piroxicam, lornoxicam, meloxicam, tenoxicam, celecoxib, etoricoxib, lumiracoxib, parecoxib, rofecoxib, valdecoxib, nimesulide, arylalkanoic acids, 2-arylpropionic acids (profens), N-arylanthranilic acids (fenamic acids), pyrazolidine derivatives, oxicams, COX-2 inhibitors, Sulphonanilides, essential fatty acids, Minozac (2-(4-(4-methyl-6-phenylpyridazin-3-yl)piperazin-1-yl)pyrimidine dihydrochloride hydrate), and combinations thereof.

In another embodiment, the anti-Alzheimer's agent is selected from the group consisting of tacrine (Cognex), donepezil (Aricept), rivastigmine (Exelon) galantamine (Reminyl), physostigmine, neostigmine, Icopezil (CP-118954, 5,7-dihydro-3-[2-[1-(phenylmethyl)-4-piperidinyl]ethyl]-6H-pyrrolo-[4,5-f]-1,2-benzisoxazol-6-one maleate), ER-127528 (4-[(5,6-dimethoxy-2-fluoro-1-indanon)-2-yl]methyl-1-(3-fluorobenzyl)piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), Metrifonate (T-588; (−)-R-α-[[2-(dimethylamino)ethoxy]methyl] benzo[b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f]oxepine-10-methanamine), SDZ-220-581 ((S)-α-amino-5-(phosphonomethyl)-[1,1′-biphenyl]-3-propionic acid), memantine (Namenda/Exiba) and 1,3,3,5,5-pentamethylcyclohexan-1-amine (Neramexane), tarenflurbil (Flurizan), tramiprosate (Alzhemed), clioquinol, PBT-2 (an 8-hydroxyquinilone derivative), 1-(2-(2-Naphthyl)ethyl)-4-(3-trifluoromethylphenyl)-1,2,3,6-tetrahydropyridine, Huperzine A, posatirelin, leuprolide or derivatives thereof, ispronicline, (3-aminopropyl)(n-butyl)phosphinic acid (SGS-742), N-methyl-5-(3-(5-isopropoxypyridinyl))-4-penten-2-amine (ispronicline), 1-decanaminium, N-(2-hydroxy-3-sulfopropyl)-N-methyl-N-octyl-, inner salt (zt-1), salicylates, aspirin, amoxiprin, benorilate, choline magnesium salicylate, diflunisal, faislamine, methyl salicylate, magnesium salicylate, salicyl salicylate, diclofenac, aceclofenac, acemetacin, bromfenac, etodolac, indometacin, nabumetone, sulindac, tolmetin, ibuprofen, carprofen, fenbufen, fenoprofen, flurbiprofen, ketoprofen, ketorolac, loxoprofen, naproxen, tiaprofenic acid, suprofen, mefenamic acid, meclofenamic acid, phenylbutazone, azapropazone, metamizole, oxyphenbutazone, sulfinprazone, piroxicam, lornoxicam, meloxicam, tenoxicam, celecoxib, etoricoxib, lumiracoxib, parecoxib, rofecoxib, valdecoxib, nimesulide, arylalkanoic acids, 2-arylpropionic acids (profens), N-arylanthranilic acids (fenamic acids), pyrazolidine derivatives, oxicams, COX-2 inhibitors, sulphonanilides, essential fatty acids, Minozac (2-(4-(4-methyl-6-phenylpyridazin-3-yl)piperazin-1-yl)pyrimidine dihydrochloride hydrate), and combinations thereof.

In yet another embodiment, the therapeutic agent capable of increasing utilization of lipids is selected from the group consisting of a PPAR-gamma agonist, a PPAR-alpha agonist, an hydroxymethylglutaryl coenzyme. A reductase inhibitor, a microsomal triglyceride transfer protein/apolipoprotein B secretion inhibitor, a cholesteryl ester transfer protein inhibitor, a squalene synthetase inhibitor, a squalene epoxidase inhibitor, a squalene cyclase inhibitor, acyl CoA-cholesterol acyltransferase inhibitor, acetyl-CoA carboxylase inhibitor and combinations thereof.

NSAIDs function, in part, as PPAR-gamma agonists. Increasing PPAR-gamma activity increases the expression of genes associated with fatty acid metabolism such as FATP (for review see (Gelman, Fruchart et al. 1999)). Accordingly, a combination of MCT and PPAR-gamma agonists will prove beneficial to individuals with decreased neuronal metabolism. In a preferred embodiment the PPAR-gamma agonist is an NSAID.

Accordingly, in another embodiment, the agent capable of increasing utilization of lipids is a PPAR agonist. Any PPAR agonist may be used as the second compound in the combination aspect of this invention. The term agonist refers to agents that activate peroxisome proliferator activator receptor activity in mammals, particularly humans. Thus, it is believed that such compounds, by activating the PPAR receptor stimulate transcription of key genes involved in fatty acid oxidation and also those involved in high density lipoprotein (HDL) assembly (for increasing HDL cholesterol). Particular agonists are PPAR-α agonist and a suitable PPAR-α agonist is, e.g., fenofibrate.

In one embodiment, the agent capable of increasing utilization of lipids is selected from the group consisting of muraglitazar, tesaglitazar, a fibrate drug, a statin, and combinations thereof.

In another embodiment, the agent capable of increasing utilization of lipids is a fibrate drug. Fibrates, such as bezafibrate, ciprofibrate, fenofibrate and Gemfibrozil, are a class of lipid lowering drugs. They act as PPAR-alpha agonists and similar to statins they increase lipoprotein lipase, apoAI and apoAII transcription and reduce levels of apoCIII. As such they have a major impact on levels of triglyceride rich lipoproteins in the plasma, presumably by increasing the use of fatty acids by peripheral tissues. Accordingly, the present invention discloses that fibrates alone or in combination with MCT would prove beneficial to patients with reduced neuronal metabolism such as those with Alzheimer's disease. The fibrate drug, in one embodiment, is selected from the group consisting of clofibrate, gemfibrozil, ciprofibrate, bezafibrate, fenofibrate, and combinations thereof.

Another embodiment of the invention provides statins as the agent capable of increasing utilization of lipids. Statins are a class of drugs with pleiotropic effects, the best characterized being inhibition of the enzyme 3-hydroxy-3-methylglutaryl CoA reductase, a key rate step in cholesterol synthesis. Statins also have other physiologic affects such as vasodilatory, anti-thrombotic, antioxidant, anti-proliferative, anti-inflammatory and plaque stabilizing properties. Additionally, statins cause a reduction in circulating triglyceride rich lipoproteins by increasing the levels of lipoprotein lipase while also decreasing apolipoprotein C-III (an inhibitor of lipoprotein lipase) (Schoonjans, Peinado-Onsurbe et al. 1999). Accordingly, administration of statins results in increased fatty acid usage, which can act synergistically with MCT administration. This should prove especially beneficial to ApoE4 carriers. One embodiment of this invention would be combination therapy consisting of statins and MCT. The statin drug includes atorvastatin, fluvastatin, lovastatin, pravastatin, simvastatin, and combinations thereof.

Caffeine and ephedra alkaloids are commonly used in over the counter dietary supplements. Ephedra alkaloids are commonly derived from plant sources such as ma-huang (Ephedra sinica). The combination of caffeine and ephedra stimulate the use of fat. Ephedra alkaloids are similar in structure to adrenaline and activate beta-adenergic receptors on cell surfaces. These adenergic receptors signal through cyclic AMP (cAMP) to increase the use of fatty acids. cAMP is normally degraded by phosphodiesterase activity. One of the functions of caffeine is to inhibit phosphodiesterase activity and thereby increase cAMP mediated signaling. Therefore caffeine potentiates the activity of the ephedra alkaloids. Accordingly, the present invention discloses that ephedra alkaloids alone can provide a treatment or prevention for conditions of reduced neuronal metabolism. Additionally, it is disclosed that ephedra alkaloids in combination with caffeine can provide a treatment or prevention for conditions of reduced neuronal metabolism. Accordingly, it is disclosed that a combination of MCT with ephedra, or MCT with caffeine, or MCT, ephedra alkaloids and caffeine together can provide a treatment or prevention for conditions of reduced neuronal metabolism.

In one embodiment, the agent capable of increasing utilization of lipids includes a cholesterol absorption inhibitor. Any cholesterol absorption inhibitor is appropriate for the present invention. The term cholesterol absorption inhibition refers to the ability of a compound to prevent cholesterol contained within the lumen of the intestine from entering into the intestinal cells and/or passing from within the intestinal cells into the blood stream. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480.

In one embodiment, the agent capable of increasing utilization of lipids includes an HMG-CoA absorption inhibitor. Any HMG-CoA reductase inhibitor may be used as the second compound in the combination aspect of this invention. The term HMG-CoA reductase inhibitor refers to compounds which inhibit the bioconversion of hydroxymethylglutaryl-coenzyme A to mevalonic acid catalyzed by the enzyme HMG-CoA reductase. Such inhibition is readily determined by those skilled in the art according to standard assays (e.g., Meth. Enzymol. 1981; 71:455-509 and references cited therein). A variety of these compounds are described and referenced below however other HMG-CoA reductase inhibitors will be known to those skilled in the art. U.S. Pat. No. 4,231,938 discloses certain compounds isolated after cultivation of a microorganism belonging to the genus Aspergillus, such as lovastatin. Also, U.S. Pat. No. 4,444,784 discloses synthetic derivatives of the aforementioned compounds, such as simvastatin. Also, U.S. Pat. No. 4,739,073 discloses certain substituted indoles, such as fluvastatin. Also, U.S. Pat. No. 4,346,227 discloses ML-236B derivatives, such as pravastatin. In addition, EP-491226A discloses certain pyridyldihydroxyheptenoic acids, such as rivastatin. In addition, U.S. Pat. No. 5,273,995 discloses certain 6-[2-(substituted-pyrrol-1-yl)alkyl]pyran-2-ones such as atorvastatin and the hemicalcium salt thereof (Lipitor™). Additional HMG-CoA reductase inhibitors include rosuvastatin, itavostatin and cerivastatin.

In one embodiment, the agent capable of increasing utilization of lipids includes a MTP/Apo B secretion (microsomal triglyceride transfer protein and/or apolipoprotein B secretion) inhibitor. Any MTP/Apo B secretion (microsomal triglyceride transfer protein and/or apolipoprotein B secretion) inhibitor may be used as the second compound in the combination aspect of this invention. The term MTP/Apo B secretion inhibitor refers to compounds which inhibit the secretion of triglycerides, cholesteryl ester, and phospholipids. Such inhibition is readily determined by those skilled in the art according to standard assays (e.g., Wetterau, J. R. 1992; Science 258:999). A variety of these compounds are known to those skilled in the art, including those disclosed in WO 96/40640 and WO 98/23593.

In one embodiment, the agent capable of increasing utilization of lipids includes a HMG-CoA synthase inhibitor. Any HMG-CoA synthase inhibitor (or HMG-CoA synthase gene expression inhibitor) may be used as the second compound in the combination aspect of this invention. The term HMG-CoA synthase inhibitor refers to compounds which inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by those skilled in the art according to standard assays (e.g., Meth Enzymol. 1975; 35:155-160: Meth. Enzymol. 1985; 110:19-26 and references cited therein). A variety of these compounds are described and referenced below, however other HMG-CoA synthase inhibitors will be known to those skilled in the art. U.S. Pat. No. 5,120,729 discloses certain beta-lactam derivatives. U.S. Pat. No. 5,064,856 discloses certain spiro-lactone derivatives prepared by culturing a microorganism (MF5253). U.S. Pat. No. 4,847,271 discloses certain oxetane compounds such as 11-(3-hydroxymethyl-4-oxo-2-oxetayl)-3,5,7-trimethyl-2,4-undeca-dienoic acid derivatives.

In one embodiment, the agent capable of increasing utilization of lipids includes an agent which decreases HMG-CoA reductase gene expression. Any compound that decreases HMG-CoA reductase gene expression may be used as the second compound in the combination aspect of this invention. These agents may be HMG-CoA reductase transcription inhibitors that block or decrease the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such regulation is readily determined by those skilled in the art according to standard assays (e.g., Meth. Enzymol. 1985; 110:9-19). Inhibitors of HMG-CoA reductase gene expression will be known to those skilled in the art, for example, U.S. Pat. No. 5,041,432 discloses certain 15-substituted lanosterol derivatives. Other oxygenated sterols that suppress synthesis of HMG-CoA reductase are discussed by E. I. Mercer (Prog. Lip. Res. 1993; 32:357-416).

In one embodiment, the agent capable of increasing utilization of lipids includes an agent which decreases CETP activity. Any compound having activity as a CETP inhibitor can serve as the second compound in the combination therapy aspect of the instant invention. The term CETP inhibitor refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art, for example, those disclosed in U.S. Pat. No. 6,140,343 and U.S. application Ser. No. 09/391,152. U.S. Pat. No. 5,512,548 discloses certain polypeptide derivatives having activity as CETP inhibitors, while certain CETP-inhibitory rosenonolactone derivatives and phosphate-containing analogs of cholesteryl ester are disclosed in J. Antibiot., 49(8): 815-816 (1996), and Bioorg. Med. Chem. Lett.; 6:1951-1954 (1996), respectively.

In one embodiment, the agent capable of increasing utilization of lipids includes an agent which decreases squalene synthetase activity. Any squalene synthetase inhibitor may be used as the second compound of this invention. The term squalene synthetase inhibitor refers to compounds which inhibit the condensation of 2 molecules of farnesylpyrophosphate to form squalene, catalyzed by the enzyme squalene synthetase. Such inhibition is readily determined by those skilled in the art according to standard assays (e.g., Meth. Enzymol. 1969; 15: 393-454 and Meth. Enzymol. 1985; 110:359-373 and references contained therein). A variety of these compounds are known to those skilled in the art, for example, U.S. Pat. No. 5,026,554 discloses fermentation products of the microorganism MF5465 (ATCC 74011) including zaragozic acid. A summary of other squalene synthetase inhibitors has been compiled (Curr. Op. Ther. Patents (1993) 861-4).

In one embodiment, the agent capable of increasing utilization of lipids includes an agent which decreases squalene epoxidase activity. Any squalene epoxidase inhibitor may be used as the second compound in the combination aspect of this invention. The term squalene epoxidase inhibitor refers to compounds which inhibit the bioconversion of squalene and molecular oxygen into squalene-2,3-epoxide, catalyzed by the enzyme squalene epoxidase. Such inhibition is readily determined by those skilled in the art according to standard assays (e.g., Biochim. Biophys. Acta 1984; 794:466-471). A variety of these compounds are known to those skilled in the art, for example, U.S. Pat. Nos. 5,011,859 and 5,064,864 disclose certain fluoro analogs of squalene. EP publication 395,768 A discloses certain substituted allylamine derivatives. PCT publication WO 9312069 A discloses certain amino alcohol derivatives. U.S. Pat. No. 5,051,534 discloses certain cyclopropyloxy-squalene derivatives.

In one embodiment, the agent capable of increasing utilization of lipids includes an agent which decreases squalene cyclase activity. Any squalene cyclase inhibitor may be used as the second component in the combination aspect of this invention. The term squalene cyclase inhibitor refers to compounds which inhibit the bioconversion of squalene-2,3-epoxide to lanosterol, catalyzed by the enzyme squalene cyclase. Such inhibition is readily determined by those skilled in the art according to standard assays (e.g., FEBS Lett. 1989; 244:347-350). Squalene cyclase inhibitors are known to those skilled in the art. For example, PCT publication W09410150 and French patent publication 2697250 disclose squalene cyclase inhibitors.

In one embodiment, the agent capable of increasing utilization of lipids includes an agent which decreases combined squalene epoxidase/squalene cyclase activity. Any combined squalene epoxidase/squalene cyclase inhibitor may be used as the second component in the combination aspect of this invention. The term combined squalene epoxidase/squalene cyclase inhibitor refers to compounds that inhibit the bioconversion of squalene to lanosterol via a squalene-2,3-epoxide intermediate. In some assays it is not possible to distinguish between squalene epoxidase inhibitors and squalene cyclase inhibitors. However, these assays are recognized by those skilled in the art. Thus, inhibition by combined squalene epoxidase/squalene cyclase inhibitors is readily determined by those skilled in art according to the aforementioned standard assays for squalene cyclase or squalene epoxidase inhibitors. A variety of squalene epoxidase/squalene cyclase inhibitors are known to those skilled in the art. U.S. Pat. Nos. 5,084,461 and 5,278,171 disclose certain azadecalin derivatives. EP publication 468,434 discloses certain piperidyl ether and thio-ether derivatives such as 2-(1-piperidyl)pentyl isopentyl sulfoxide and 2-(1-piperidyl)ethyl ethyl sulfide. PCT publication WO 9401404 discloses certain acyl-piperidines such as 1-(1-oxopentyl-5-phenylthio)-4-(2-hydroxy-1-methyl)-ethyl)piperidine. U.S. Pat. No. 5,102,915 discloses certain cyclopropyloxy-squalene derivatives.

In one embodiment, the agent capable of increasing utilization of lipids includes an agent that decreases ACAT activity. Any ACAT inhibitor can serve as the second compound in the combination therapy aspect of this invention. The term ACAT inhibitor refers to compounds that inhibit the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research., 24:1127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity.

Any anti-atherosclerosis agent may be used as the therapeutic agent of the invention. In one embodiment, the anti-atherosclerotic agent includes an anti-platelet/anti-thrombotic agent, estrogen receptor modulator, an anti-cholesterolemia agent, and combinations thereof. For example, they may be used in combination with cholesterol synthesis inhibitors, fibrates, niacin, garlic extract, ion-exchange resins, antioxidants and bile acid sequestrants. Any anti-platelet and anti-thrombotic agent may be used as the second compound in the combination aspect of this invention. Suitable anti-platelet and anti-thrombotic agents include, e.g., tPA, uPA, warfarin, hirudin, hirulog, and other thrombin inhibitors, heparin, heparinoids and thromboplastin activating factor inhibitors. Other compounds that are marketed for hyperlipidemia, including hypercholesterolemia and which are intended to help prevent or treat atherosclerosis include bile acid sequestrants, such as Welchol™ (colesevalam HCl), Colestid™ (colestipol HCl), LoCholeSt™ and Questran™ (cholestyramine); and fibric acid derivatives, such as Atromid™ (clofibrate), Lopid™ (gemfibrozil) and Tricor™ or Lofibra™ (fenofibrate).

Any estrogen receptor modulator, estrogen agonist or estrogen antagonist may be used as the second compound in the combination aspect of this invention. Such compounds are known to mediate lipid levels. Suitable estrogen receptor modulators, estrogen agonists or estrogen antagonists include the compounds disclosed in International Patent Application Publication No. WO96/21656 and U.S. Pat. No. 5,552,412. Preferred such compounds include raloxifene, lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof.

Any anti-diabetic agent may be used as the therapeutic agent of the invention. In one embodiment, the anti-diabetic agent includes the following classes of compounds: glycogen phosphorylase inhibitors, aldose reductase inhibitors, sorbitol dehydrogenase inhibitors, glucosidase inhibitors, amylase inhibitors, a phosphodiesterase inhibitor, a protein kinase C-beta inhibitor, a PTB1B inhibitor, a glucagons antagonist, a glycogen synthase kinase-3 inhibitor, a GLP-1 agonist, a soluble guanylate cyclase activator, and combinations thereof. Specific anti-diabetic agents include sulfonyl urea, a biguanide, a thiazolidinedione, a meglitinide, and combinations thereof.

In one embodiment, the anti-diabetic agent is a glycogen phosphorylase inhibitor. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays (e.g., J. Med. Chem. 41 (1998) 2934-2938). A variety of glycogen phosphorylase inhibitors are known to those skilled in the art including those described in WO 96/39384 and WO 96/39385.

In one embodiment, the anti-diabetic agent is an aldose reductase inhibitor. The term aldose reductase inhibitor refers to compounds that inhibit the bioconversion of glucose to sorbitol, which is catalyzed by the enzyme aldose reductase. Aldose reductase inhibition is readily determined by those skilled in the art according to standard assays (e.g., J. Malone, Diabetes, 29:861-864 (1980). “Red Cell Sorbitol, an Indicator of Diabetic Control”). A variety of aldose reductase inhibitors are known to those skilled in the art such as zopolrestat, epalrestat, ponalrestat, zenarestat and fidarestat.

In one embodiment, the anti-diabetic agent is a sorbitol dehydrogenase inhibitor. The term sorbitol dehydrogenase inhibitor (SDI) refers to compounds that inhibit the bioconversion of sorbitol to fructose which is catalyzed by the enzyme sorbitol dehydrogenase. Such sorbitol dehydrogenase inhibitor activity is readily determined by those skilled in the art according to standard assays (e.g., Analyt. Biochem (2000) 280: 329-331). A variety of sorbitol dehydrogenase inhibitors are known, for example, U.S. Pat. Nos. 5,728,704 and 5,866,578 disclose compounds and a method for treating or preventing diabetic complications by inhibiting the enzyme sorbitol dehydrogenase. Other SDIs include those dislcosed in International Patent Application Publication No. WO00/59510. A particularly preferred SDI is 1R-(4-(4-(4,6-dimethyl)-[1,3,5]triazin-2-yl)-2R,6S-dimethyl-piperazin-1-yl)-pyrimidin-2-yl)-ethanol.

In one embodiment, the anti-diabetic agent is a glucosidase inhibitor. A glucosidase inhibitor inhibits the enzymatic hydrolysis of complex carbohydrates by glycoside hydrolases, for example amylase or maltase, into bioavailable simple sugars, for example, glucose. The rapid metabolic action of glucosidases, particularly following the intake of high levels of carbohydrates, results in a state of alimentary hyperglycemia which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known chyme remaining in the stomach promotes the production of gastric juice, which initiates or favors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969) 8: 4214).

A generally preferred glucosidase inhibitor comprises an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistat, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethyl)-D-epi-inositol, and the various N-substituted pseudo-aminosugars related thereto, are disclosed in U.S. Pat. No. 4,701,559. The glucosidase inhibitor, miglitol, (2R,3R,4R,5S)-1-(2-hydroxyethyl)-2-(hydroxymethyl)-3,4,5-piperidinetriol, and the various 3,4,5-trihydroxypiperidines related thereto, are disclosed in U.S. Pat. No. 4,639,436. The glucosidase inhibitor, emiglitate, ethyl p-[2-[(2R,3R,4R,5S)-3,4,5-trihydroxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-O—O-β-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-[(2R,3R,4R,5S)-3,4,5-trihydroxy-2-(hydroxymethyl)piperidino]-α-D-glucopyranoside sesquihydrate, the deoxy-nojirimycin derivatives related thereto, the various pharmaceutically acceptable salts thereof and synthetic methods for the preparation thereof, are disclosed in U.S. Pat. Nos. 5,157,116 and 5,504,078. The glycosidase inhibitor, salbostatin and the various pseudosaccharides related thereto, are disclosed in U.S. Pat. No. 5,091,524. A variety of amylase inhibitors are known to one of ordinary skill in the art. The amylase inhibitor, tendamistat and the various cyclic peptides related thereto, are disclosed in U.S. Pat. No. 4,451,455. The amylase inhibitor Al-3688 and the various cyclic polypeptides related thereto are disclosed in U.S. Pat. No. 4,623,714. The amylase inhibitor, trestatin, consisting of a mixture of trestatin A, trestatin B and trestatin C and the various trehalose-containing aminosugars related thereto are disclosed in U.S. Pat. No. 4,273,765.

In one embodiment, the anti-diabetic agent is a phosphodiesterase (PDE) inhibitor. Any PDE5 or PDE11 inhibitor may be used as the second compound of a combination of this invention. It is particularly preferred that a PDE5 inhibitor be used as the second compound of this invention. Suitable PDE5 inhibitors include the pyrazolo[4,3-d]pyrimidin-7-ones disclosed in EP-A-0463756; the pyrazolo[4,3-d]pyrimidin-7-ones disclosed in EP-A-0526004; the pyrazolo[4,3-d]pyrimidin-7-ones disclosed in published international patent application WO 93/06104; the isomeric pyrazolo[3,4-d]pyrimidin-4-ones disclosed in International Patent Application Publication No. WO93/07149; the quinazolin-4-ones disclosed in International Patent Application Publication No. WO93/12095; the pyrido[3,2-d]pyrimidin-4-ones disclosed in International Patent Application Publication No. WO94/05661; the purin-6-ones disclosed in International Patent Application Publication No. WO94/00453; the pyrazolo[4,3-d]pyrimidin-7-ones disclosed in International Patent Application Publication No. WO98/49166; the pyrazolo[4,3-d]pyrimidin-7-ones disclosed in International Patent Application Publication No. WO99/54333; the pyrazolo[4,3-d]pyrimidin-4-ones disclosed in EP-A-0995751; the pyrazolo[4,3-d]pyrimidin-7-ones disclosed in International Patent Application Publication No. WO00/24745; the pyrazolo[4,3-d]pyrimidin-4-ones disclosed in EP-A-0995750; the compounds disclosed in International Patent Application Publication No. WO95/19978; the compounds disclosed in International Patent Application Publication No. WO99/24433; the pyrazolo[4,3-d]pyrimidin-7-ones disclosed in International Patent Application Publication No. WO01/27112; the pyrazolo[4,3-d]pyrimidin-7-ones disclosed in International Patent Application Publication No. WO01/27113; the compounds disclosed in EP-A-1092718; the compounds disclosed in EP-A-1092719; and the compounds disclosed in International Patent Application Publication No. WO93/07124. Preferred PDE5 inhibitors for use as a second compound in a combination of this invention include: 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); 6-benzo[1,3]dioxol-5-yl-2-methyl-2,3,6,7,12,12a-hexahydro-pyrazino[1′,2′:1,6]pyrido[3,4-b]indole-1,4-dione (cialis); (+)-3-ethyl-5-[5-(4-ethylpiperazin-1-ylsulphonyl)-2-(2-methoxy-1(R)-methyl ethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-m ethoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO01/27113, Example 8); 5-[2-iso-butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO01/27113, Example 15); 5-[2-ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO01/27113, Example 66); 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one (see WO01/27112, Example 124); 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one (see WO01/27112, Example 132); (6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; the compound of example 11 in WO93/07124 (EISAI); and compounds 3 and 14 from Rotella D P, J. Med. Chem., 2000, 43, 1257.

Other anti-diabetic agents that may be used as the second compound of a combination of this invention include protein kinase C-β inhibitors, PTP1B inhibitor, glucagon antagonists, glycogen synthase kinase-3 (GSK-3) inhibitors, GLP-1 agonists, vanadyl sulfate, chromium picolinate, vitamin E, or soluble guanylate cyclase (sGC) activator.

In another embodiment, the therapeutic agent is an anti-obesity agent. Any anti-obesity agent may be used. Such anti-obesity activity is readily determined by those skilled in the art according to standard assays (e.g., as detailed below). General classes of anti-obesity agents include a thyromimetic, a melanocortin receptor modulator, a serotonin receptor agonist, a neurokinin receptor antagonist, a modulator of transporters of noradrenaline or dopamine, a beta-adrenergic agonist, a NPY receptor antagonist, and combinations thereof. Any thyromimetic may be used as the therapeutic agent. Such thyromimetic activity is readily determined by those skilled in the art according to standard assays (e.g., Atherosclerosis (1996) 126: 53-63). A variety of thyromimetic agents are known to those skilled in the art, for example those disclosed in U.S. Pat. Nos. 4,766,121; 4,826,876; 4,910,305; 5,061,798; 5,284,971; 5,401,772; 5,654,468; and 5,569,674. Other antiobesity agents include sibutramine which can be prepared as described in U.S. Pat. No. 4,929,629, and bromocriptine which can be prepared as described in U.S. Pat. Nos. 3,752,814 and 3,752,888. Any melanocortin receptor agonist, melanocortin receptor modulator or melanocortin receptor enhancer may be used as the anti-obesity agent. Suitable melanocortin receptor agonists, modulators or enhancers include melanotan II; and compounds disclosed in International Patent Application Publication Nos. WO99/64002, WO0/74679, WO99/55679, WO01/05401, WO0/58361, WO01/14879, WO01/13112 and WO99/54358. Any serotonin receptor agonist, antagonist or modulator may be used as the anti-obesity agent of this invention. It is particularly preferred to use agonists, antagonists or modulators of 5HT1A. Suitable agonists, antagonists or modulators include 5HT2A; 5HT2C; 5HT3; and 5HT6 receptors, including those described in International Patent Application Publication Nos. WO99/02159, WO00/02550 and WO00/28993. Any neurokinin receptor (NK) antagonist may be used as the anti-obesity agent of this invention. Suitable NK receptor antagonists include those described in International Patent Application Publication No. WO99/64008. Any modulator of transporters for noradrenaline or dopamine may be used as the anti-obesity agent of this invention. Suitable such modulators include bupropion.

Any β-adrenergic agonist may be used as the anti-obesity agent of this invention. β-adrenergic agonist agents have been categorized into β1, β2, and β3 subtypes. Agonists of β-receptors promote the activation of adenyl cyclase. Activation of β1 receptors invokes increases in heart rate. Activation of β2 receptors induces relaxation of smooth muscle tissue which produces a drop in blood pressure and the onset of skeletal muscle tremors. Activation of β3 receptors is known to stimulate lipolysis, which is the breakdown of adipose tissue triglycerides to glycerol and fatty acids. Activation of β3 receptors also stimulates the metabolic rate, thereby increasing energy expenditure. Such activity is readily determined by those skilled in the art according to standard assays. Several compounds are described and referenced below; however, other β-adrenergic agonists will be known to those skilled in the art. International Patent Application, Publication No. WO 96/35671 (the disclosure of which is incorporated herein by reference) discloses compounds, such as substituted aminopyridines, which are β-adrenergic agonists. International Patent Application, Publication No. 93/16189 (the disclosure of which is incorporated herein by reference) discloses the use of selective β3 receptor agonists in combination with compounds which modify eating behavior for the treatment of obesity.

Any NPY receptor antagonist may be used as the anti-obesity agent of this invention. The term NPY receptor antagonist refers to compounds which interact with NPY receptors and inhibit the activity of neuropeptide Y at those receptors and thus are useful in treating disorders associated with neuropeptide Y, such as feeding disorders, including obesity. Such inhibition is readily determined by those skilled in the art according to standard assays (such as those described in International Patent Application, Publication No. WO 99/07703). In addition, the compounds described and referenced below are NPY receptor antagonists; however, other NPY receptor antagonists will also be known to those skilled in the art. WO 99/07703 (the disclosure of which is hereby incorporated by reference) discloses certain 4-aminopyrrole (3,2-d) pyrimidines as neuropeptide Y receptor antagonists. WO 96/14307, WO 96/40660, WO 98/03492; WO 98/03494; WO 98/03493; WO 96/14307; WO 96/40660, (the disclosures of which are hereby incorporated by reference) disclose additional compounds, such as substituted benzylamine derivatives, which are useful as neuropeptide Y specific ligands.

Other anti-obesity agents for use in the present invention include phenylpropanolamine, ephedrine, pseudoephedrine, a cholecystokinin-A (hereinafter referred to as CCK-A) agonist, a monoamine reuptake inhibitor (such as sibutramine), a sympathomimetic agent, a dopamine agonist (such as bromocriptine), a melanocyte-stimulating hormone receptor agonist or mimetic, a melanocyte-stimulating hormone analog, a cannabinoid receptor antagonist, a melanin concentrating hormone antagonist, the OB protein (hereinafter referred to as “leptin”), a leptin analog, or a galanin antagonist. Other anti-obesity agents include phosphatase 1B inhibitors, bombesin agonists, dehydroepiandrosterone or analogs thereof, glucocorticoid receptor modulators, orexin receptor antagonists, urocortin binding protein antagonists, glucagon-like peptide-1 (insulinotropin) agonists or dipeptidyl peptidase IV (DPPIV) inhibitors. A particularly preferred monoamine reuptake inhibitor is sibutramine, which can be prepared as disclosed in U.S. Pat. No. 4,929,629, the disclosure of which is incorporated herein by reference. A particularly preferred dopamine agonist is bromocriptine, which can be prepared as disclosed in U.S. Pat. Nos. 3,752,814 and 3,752,888, the disclosures of which are incorporated herein by reference. Another preferred anorectic agent is phentermine, which can be prepared as disclosed in U.S. Pat. No. 2,408,345, the disclosure of which is incorporated herein by reference.

Therapeutic agents of the present invention also include other cardiovascular (e.g., anti-hypertensive agents). Such anti-hypertensive activity is readily determined by those skilled in the art according to standard assays (e.g., blood pressure measurements). Any α-adrenergic receptor antagonist compound may be used as the anti-hypertensive agent of this invention. Suitable α-adrenergic receptor antagonists for use herein include the α-adrenergic receptor blockers described in International Patent Application Publication No. WO99/30697. Selective α1-adrenoceptor, α2, α2-adrenoceptor blockers and non-selective adrenoceptor blockers may also be used as the second α-adrenergic receptor antagonist compound of this invention. Suitable α1-adrenoceptor blockers include phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, doxazosin, terazosin, abanoquil and prazosin. Suitable α2-adrenoceptor blockers include those disclosed in U.S. Pat. No. 6,037,346, dibenarnine, tolazoline, trimazosin and dibenarnine. Suitable α-adrenergic receptors for use as the anti-hypertensive agent of this invention are also described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000. Other suitable α2-adrenoceptor blockers include clonidine, papaverine, papaverine hydrochloride, each of which may optionally be administered in the presence of a cariotonic agent such as, but not limited to, pirxamine.

Any nitrous oxide donor (NO-donor or NO-agonist) compound may be used as the anti-hypertensive agent of this invention. Suitable NO-donor compounds include organic nitrates, such as mono-, di- or tri-nitrates; organic nitrate esters such as glyceryl binitrate (also known as nitroglycerin), isosorbide 5-mononitrate, isosorbide dinitrate, pentaerythritol tetranitrate, erythrityl tetranitrate, amylnitrate, a diazenium diolate (NONOate), and 1,5-pentanedinitrate; sodium nitroprusside (SNP); 3-morpholinosydnonimine molsidomine; S-nitroso-N-acetyl penicilliamine (SNAP); S-nitroso-N-glutathione (SNO-GLU); N-hydroxy-L-arginine; linsidomine; linsidomine chlorohydrate; (SIN-1) S-nitroso-N-cysteine; L-arginine; ginseng; zizphi fructus; molsidomine; and nitrosylated maxisylyte derivatives such as NMI-678-11 and NMI-937 (International Patent Application Publication No. WO00/12075). Any potassium channel opener or modulator may be used as anti-hypertensive agent of this invention. Suitable potassium channel openers/modulators for use herein include nicorandil, cromokalim, levcromakalim, lemakalim, pinacidil, cliazoxide, minoxidil, charybdotoxin, glyburide, 4-aminopyridine and barium chloride (BaCl₂). Any vasodilator agent may be used as anti-hypertensive agent of this invention. Suitable vasodilator agents for use herein include nimodepine, pinacidil, cyclandelate, isoxsuprine, chloroprumazine, halo peridol and trazodone. Any ergot alkoloid may be used as the anti-hypertensive agent of this invention. Suitable ergot alkaloids include those disclosed in U.S. Pat. No. 6,037,346; acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride. Any angiotensin receptor antagonist may be used as anti-hypertensive agent of this invention. Suitable angiotensin receptor antagonists include losartan, candersartan, eprosartan, irbesartan and valsartan. Any substrate for NO-synthase may be used as the anti-hypertensive agent of this invention. Suitable NO-synthase substrates include, inter alia, L-arginine. Any calcium channel blocker may be used as anti-hypertensive agent of this invention. Suitable calcium channel blockers include, amlodipine and amlodipine besylate (also known as Norvasc). Specific examples of antihypertensive agents include calcium channel blockers, such as Cardizem™, Dilacor™ or Tiazac™ (diltiazem HCl), Adalat™ or Procardia™ (nifedipine), Calan™, Covera™, Verelan™, Isoptin™ (verapamil HCl), Cardene™ (nicardipine), DynaCirc™ (isradipine), Sular™ (nisoldipine), Vascor™ (bepridil), Nimotop™ (nimodipine), Norvasc™ (amlodipine besylate), and Plendil™ (felodipine).

Any angiotensin converting enzyme inhibitor (ACE inhibitor) may be used as anti-hypertensive agent of this invention. Suitable ACE inhibitors include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361. Any compound which is a combined inhibitor of angiotensin-converting enzyme and neutral endopeptidase may be used as anti-hypertensive agent of this invention. A suitable such combined inhibitor is, e.g., omapatrilat.

The present invention also provides a composition further comprising a medium chain triglyceride and an essential fatty acid therapeutic agent which includes a triglyceride containing essential fatty acids, such as; α-Linolenic acid (18:3), Linoleic acid (18:2), eicosapentaenoic acid or EPA (20:5), docosahexaenoic acid or DHA (22:6), gamma-linolenic acid or GLA (18:3), dihomo-gamma-linolenic acid or DGLA (20:3), arachidonic acid or AA (20:4), and mixtures thereof. The essential fatty acid may also comprise the free fatty acids or the phospholipids of the fatty acids referenced above. The first and second composition may also be administered with an essential fatty acid therapeutic agent.

From the description above, a number of advantages of the invention for treating and preventing Alzheimer's Disease become evident:

Prior art on AD has largely focused on prevention and clearance of amyloid deposits. The role of these deposits in AD remains controversial and may only be a marker for some other pathology. The present invention provides a novel route for treatment and prevention of AD based on alleviating the reduced neuronal metabolism associated with AD, and not with aspects of amyloid accumulation.

Current treatments for AD are merely palliative and do not address the reduced neuronal metabolism associated with AD. Ingestion of medium chain triglycerides as a nutritional supplement is a simple method to provide neuronal cells, in which glucose metabolism is compromised, with ketone bodies as a metabolic substrate.

Increased blood levels of ketone bodies can be achieved by a diet rich in medium chain triglycerides.

Medium chain triglycerides can be infused intravenously into patients or administered orally.

(e) Levels of ketone bodies can be easily measured in urine or blood by commercially available products (e.g., Ketostix®, Bayer, Inc.).

In one embodiment, the compositions of the invention, such as first compositions, are in the form of food compositions. In certain embodiments, the composition is a food composition, further comprising in addition to the ketogenic compound and therapeutic agent, about 15% to about 50% protein, about 5% to about 40% fat, about 5% to about 40% carbohydrate, each on a dry weight basis, and having a moisture content of about 5% to about 20%. In certain embodiments, the foods are intended to supply complete necessary dietary requirements. Also provided are compositions that are useful as snacks, as nutrition bars, or other forms of food products or nutritional or dietary supplements, including tablets, capsules, gels, pastes, emulsions, caplets, and the like. Optionally, the food compositions can be a dry composition, a semi-moist composition, a wet composition, or any mixture thereof. In one embodiment, the food products are complete and nutritionally balanced, while in others they are intended as nutritional supplements to be used in connection with a well-balanced or formulated diet.

In one embodiment, the compositions of the invention, including the first composition, is a food supplement, such as drinking water, beverage, liquid concentrate, gel, pudding, yogurt, powder, granule, paste, suspension, chew, morsel, treat, snack, pellet, pill, capsule, tablet, or any other delivery form. In one embodiment, the nutritional supplement can be administered to the mammal in small amounts, or can be diluted before administration to the mammal. In some embodiments, the nutritional supplement comprisings of the invention may require admixing with water or the like prior to administration to the mammal, for example to adjust the dose, to make it more palatable, or to allow for more frequent administration in smaller doses. The compositions may be refrigerated or frozen, and the ketogenic compound(s) may be pre-blended with the other components of the composition to provide the beneficial amounts needed; may be emulsified, coated onto a food composition, nutritional or dietary supplement; or may be added to a composition prior to consuming it.

In one embodiment, the composition, including the first composition, comprise MCT in an amount effective for the treatment or prevention of Alzheimer's disease, mild cognitive impairment, or other disease of reduced neuronal metabolism as described elsewhere herein in the patient to which the compositions of the invention have been administered. The composition is in a range of about 1% to about 50% MCT on a dry matter basis, although a lesser or greater percentage may be applied. In various embodiments, the amount is about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%. 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 31%, 32%, 33%, 34%, 35%, 36%, 37%, 38%, 39%, 40%, 41%, 42%, 43%, 44%, 45%, 46%, 47%, 48%, 49%, or about 50%, or more, of the composition on a dry weight basis. Nutritional supplements may be formulated to contain several fold higher concentrations of MCT and therapeutic agent, to be amenable for administration in the form of a tablet, capsule, liquid concentrate, or other similar dosage form, or to be diluted before administration, such as by dilution in water, spraying or sprinkling onto food, and other similar modes of administration.

In various embodiments, the compositions optionally comprise supplementary substances such as minerals, vitamins, salts, condiments, colorants, and preservatives. Non-limiting examples of supplementary materials include calcium, phosphorous, potassium, sodium, iron, chloride, boron, copper, zinc, magnesium, manganese, iodine, selenium, and the like. Non-limiting examples of supplementary vitamins include vitamin A, any of the B vitamins, vitamin C, vitamin D, vitamin E, and vitamin K, including various salts, esters or other derivatives of the foregoing. Additional dietary supplements may also be included, for example, any form of niacin, pantothenic acid, inulin, folic acid, biotin, amino acids, and the like, as well as salts and derivatives thereof. In addition, the compositions may comprise beneficial long chain polyunsaturated fatty acids (PUFAs) such as the omega 3 and/or omega 6 fatty acids, arachidonic acid, eicosapentaenoic acid, docosapentaenoic acid, or docosahexaenoic acid, as well as combinations thereof. Optional supplementary substances also include, for example, choline, phosphatidyl serine, alpha-lipoic acid, CoQ10, acetyl-L-carnitine, and herbal extracts such as Gingko biloba, Bacopa monniera, Convolvulus pluricaulis, and Leucojum aestivum.

In various embodiments, the food or drink compositions of the invention optionally comprise, on a dry weight basis, from about 15% to about 50% crude protein. The crude protein material may comprise one or more proteins from any source whether animal, plant, or other. For example, vegetable proteins such as soybean, cottonseed, and peanut are suitable for use herein. Animal proteins such as casein, albumin, and meat protein, including pork, lamb, poultry, fish, or mixtures thereof are useful.

The compositions may further comprise, on a dry weight basis, from about 5% to about 40% fat. The compositions may further comprise a source of carbohydrate. The food compositions typically comprise from about 15% to about 40% carbohydrate, on a dry weight basis. Examples of such carbohydrates include grains or cereals such as rice, corn, sorghum, alfalfa, barley, soybeans, canola, oats, wheat, or mixtures thereof.

In certain embodiments, the compositions also comprise at least one fiber source. Any of a variety of soluble or insoluble fibers suitable for use in foods may be utilized, and such will be known to those of ordinary skill in the art. Presently included fiber sources include beet pulp (from sugar beet), gum arabic, gum talha, psyllium, rice bran, carob bean gum, citrus pulp, pectin, fructooligosacharide addition to the short chain oligofructose, mannanoligofructose, soy fiber, arabinogalactan, galactoololigosaccharide, arabinoxylan, or mixtures thereof. Additionally, probiotic microorganisms, such as Lactobacillus or Bifidobacterium species, for example, may be added to the compositions.

In another embodiment, the present invention includes a method for treatment of dementia of Alzheimer's type or mild cognitive impairment comprising the steps of: identifying a population of mammals having or at risk of having dementia of Alzheimer's type or mild cognitive impairment; dividing the population into at least a control group and one or more test groups; formulating at least one delivery system for delivering a composition comprising at least one compound capable of elevating ketone body concentrations in an amount effective for elevating at least one type of ketone body in the blood of an individual mammal; wherein, on an extended regular basis, each test group receives a formulation delivering a composition comprising i) at least one compound capable of elevating ketone body concentrations in an amount effective for the treatment of or prevention of loss of cognitive function caused by reduced neuronal metabolism in dementia of Alzheimer's type or mild cognitive impairment; and ii) a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof, capable of elevating ketone body concentrations in an amount effective for elevating at least one type of ketone body in the blood of an individual mammal, and the control group does not receive any of the above composition; comparing at least one neuropsychological test result in the control and test groups; determining which of the delivery systems for delivering the composition comprising MCT was effective in improving the results of at least one neuropsychological test; and administering a treatment-based delivery system determined in step (e) to a population of aging mammals, thereby treating the dementia of Alzheimer's type or mild cognitive impairment.

In another embodiment, the present invention includes a method of individualizing a treatment for dementia of Alzheimer's type or mild cognitive impairment, comprising: determining a patient's apolipoprotein E genotype; providing a pharmaceutical composition comprising: i) at least one compound capable of elevating ketone body concentrations in an amount effective for the treatment of or prevention of loss of cognitive function caused by reduced neuronal metabolism in dementia of Alzheimer's type or mild cognitive impairment; and ii) a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof, which provides an ketone body level effective for treatment dementia of Alzheimer's type or mild cognitive impairment for said genotype, whereupon the treatment for Alzheimer's Disease is individualized.

In another embodiment, the present invention includes a method of elevating ketone body levels comprising administering i) at least one compound capable of elevating ketone body concentrations in an amount effective for the treatment of or prevention of loss of cognitive function caused by reduced neuronal metabolism in dementia of Alzheimer's type or mild cognitive impairment; and ii) a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof, to a patient in need thereof.

In another embodiment, the present invention includes a method of increasing cognitive ability in a patient suffering from Alzheimer's Disease or Mild Cognitive Impairment, comprising administering i) at least one compound capable of elevating ketone body concentrations in an amount effective for the treatment of or prevention of loss of cognitive function caused by reduced neuronal metabolism in dementia of Alzheimer's type or mild cognitive impairment; and ii) a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof, to a patient in need thereof.

The present invention also includes a method of treating reduced neuronal metabolism comprising administering a therapeutic agent which induces utilization of fatty acids, comprising administering i) at least one compound capable of elevating ketone body concentrations in an amount effective for the treatment of or prevention of loss of cognitive function caused by reduced neuronal metabolism in dementia of Alzheimer's type or mild cognitive impairment; and ii) a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof, to a patient in need thereof.

The present invention also comprises a liquid dosage form for oral consumption comprising: i) a unit dose sufficient to a) raise blood levels of D-β-hydroxybutyrate to about 0.1 to about 5 mM or b) raise urinary excretion levels of D-β-hydroxybutyrate to about 5 mg/dL to about 160 mg/dL; a plurality of vitamins; flavoring, and a carbohydrate source and wherein the MCT are of the formula:

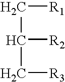

wherein the R1, R2, and R3 esterified to the glycerol backbone are each independently fatty acids having carbon chains of 5-12 carbons; and ii) a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof.

Accordingly, the reader will see that the use of medium chain triglycerides (MCT) or fatty acids as a treatment and preventative measure of Alzheimer 's disease (AD) provides a novel means of alleviating reduced neuronal metabolism associated with AD. It is the novel and significant insight of the present invention that use of MCT results in hyperketonemia which will provide increased neuronal metabolism for diseases associated with reduced neuronal metabolism, such as AD, ALS, Parkinson's Disease and Huntington's Disease. Although the description above contains many specificities, these should not be construed as limiting the scope of the invention but merely as providing illustrations for some of the presently preferred embodiments of this invention. For example, supplementation with MCT may prove more effective when combined with insulin sensitizing agents such as vanadyl sulfate, chromium picolinate, and vitamin E. Such agents may function to increase glucose utilization in compromised neurons and work synergistically with hyperketonemia. In another example MCT can be combined with compounds that increase the rates of fatty acid utilization such as L-carnitine and its derivatives. Mixtures of such compounds may synergistically increase levels of circulating ketone bodies.

Thus the scope of the invention should be determined by the appended claims and their legal equivalents, rather than by the examples given.

### REFERENCES

Throughout the specification, citations to a number of references have been made. Each of these references is incorporated by reference herein in its entirety. Many of the references are summarized here:
Beffert, U., Danik, M., Krzywkowski, P., Ramassamy, C., Berrada, F., and Poirier, J. (1998) The neurobiology of apolipoproteins and their receptors in the CNS and Alzheimer's disease. Brain Res Brain Res Rev 27:119-42.
Blass, J. P., and Zemcov, A. (1984) Alzheimer's disease. A metabolic systems degeneration? Neurochem Pathol 2:103-14.

Craft, S., Newcomer, J., Kanne, S., Dagogo-Jack, S., Cryer, P., Sheline, Y., Luby, J., Dagogo-Jack, A., and Alderson, A. (1996) Memory improvement following induced hyperinsulinemia in Alzheimer's disease. Neurobiol Aging 17:123-30.
Corbo, R. M. and Sacchi, R. (1999) Apoliporpotein E (APOE) allele distribution in the world. Is APOE*4 a ‘thrifty’ allele. Ann Hum Genet. 63:301-10.
Davis, J. N., and Chisholm, J. C. (1999). Alois Alzheimer and the amyloid debate. Nature 400:810.
Edmond, J. (1992) Energy metabolism in developing brain cells. Can J Physiol Pharmacol 70:S118-29.
Evans, D. A., Funkenstein, H. H., Albert, M. S., Scherr, P. A., Cook, N. R., Chown, M. J., Hebert, L. E., Hennekens, C. H., and Taylor, J. O. (1989) Prevalence of Alzheimer's disease in a community population of older persons. Higher than previously reported. 262:2551-6.
Finch, C. E., and Cohen, D. M. (1997) Aging, metabolism, and Alzheimer disease: review and hypotheses. Exp Neurol 143:82-102.
Frolich, L., Blum-Degen, D., Bernstein, H. G., Engelsberger, S., Humrich, J., Laufer, S., Muschner, D., Thalheimer, A., Turk, A., Hoyer, S., Zochling, R., Boissl, K. W., Jellinger, K., and Riederer, P. (1998) Brain insulin and insulin receptors in aging and sporadic Alzheimer's disease. J Neural Transm 105:423-38.
Bullock, R. (2002). “New drugs for Alzheimer's disease and other dementias.” Br J Psychiatry 180: 135-9.

Gelman, L., J. C. Fruchart, et al. (1999). “An update on the mechanisms of action of the peroxisome proliferator-activated receptors (PPARs) and their roles in inflammation and cancer.” Cell Mol Life Sci 55(6-7): 932-43.
Hertz, L., A. C. Yu, et al. (2000). “Neuronal-astrocytic and cytosolic-mitochondrial metabolite trafficking during brain activation, hyperammonemia and energy deprivation.” Neurochem Int 37(2-3): 83-102.
Schoonjans, K., J. Peinado-Onsurbe, et al. (1999). “3-Hydroxy-3-methylglutaryl CoA reductase inhibitors reduce serum triglyceride levels through modulation of apolipoprotein C-III and lipoprotein lipase.” FEBS Lett 452(3): 160-4.
Selkoe, D. J. (2001). “Alzheimer's disease: genes, proteins, and therapy.” Physiol Rev 81(2): 741-66.
Staels, B., J. Dallongeville, et al. (1998). “Mechanism of action of fibrates on lipid and lipoprotein metabolism.” Circulation 98(19): 2088-93.
Gregg, R. E., Zech, L. A., Schaefer, E. J., Stark, D., Wilson, D., and Brewer, H. B. Jr. (1986). Abnormal in vivo metabolism of apolipoprotein E4 in humans. J Clin Invest 78:815-21.
Goodman, L. S., Limbird, L. E., Milinoff, P. B., Gilman, A. G., and Hardman, J. G. (editors). (1996). The Pharmacological Basis of Therapeutics, 9.sup.th Ed., McGraw-Hill.
Hall K., Gureje O., Gao S., Ogunniyi A., Hui S. L., Baiyewu O., Unverzagt F. W., Oluwole S., Hendrie H. C. (1998) Risk factors and Alzheimer's disease: a comparative study of two communities. Aust N Z J Psychiatry 32:698-706.
Hamosh, M. (1990) In: Lingual and Gastric Lipases: Their role in fat digestion. CRC press, Boca Raton, Fla.
Hanlon C. S., and Rubinsztein D.C. (1995) Arginine residues at codons 112 and 158 in the apolipoprotein E gene correspond to the ancestral state in humans. Atherosclerosis 112:85-90.
Hasselbalch, S. G., Madsen, P. L., Hageman, L. P., Olsen, K. S., Justesen, N., Holm, S., and Paulson, O. B. (1996) Changes in cerebral blood flow and carbohydrate metabolism during acute hyperketonemia. Am J Physiol 270:E746-51.
Hertz, L., A. C. Yu, et al. (2000). “Neuronal-astrocytic and cytosolic-mitochondrial metabolite trafficking during brain activation, hyperammonemia and energy deprivation.” Neurochem Int 37(2-3): 83-102.
Hoyer, S. (1998) Is sporadic Alzheimer disease the brain type of non-insulin dependent diabetes mellitus? A challenging hypothesis. J Neural Transm 105:415-22.
Hoyer, S. (1992) Oxidative energy metabolism in Alzheimer brain. Studies in early-onset and late-onset cases. Mol Chem Neuropathol 16:207-24.
Jolles, J., Bothmer, J., Markerink, M., and Ravid, R. (1992) Phosphatidylinositol kinase is reduced in Alzheimer's disease. J Neurochem 58: 6-9.
Kolanowski, J., Young, J. B., and Landsberg L. (1994) Stimulatory influence of D(−)3-hydroxybutyrate feeding on sympathetic nervous system activity in the rat. Metabolism 43:180-5.
Klivenyi, P., Ferrante, R. J., Matthews, R. T., Bogdanov, M. B., Klein, A. M. Andreassen, O. A., Mueller, G., Wermer, M., Kaddurah-Daouk, R., and Beal, M. F. (1999) Neuroprotective effects of creatine in a transgenic animal model of amyotrophic lateral sclerosis. Nat. Med. 5:347-50.
Koo, E. H., Lansbury, P. T., Jr., and Kelly, J. W. (1999) Amyloid diseases: abnormal protein aggregation in neurodegeneration. Proc Natl Acad Sci U S A. 96:9989-90.
Knouff, C., Hinsdale, M. E., Mezdour, H., Altenburg, M. K., Watanabe, M., Quarfordt, S. H., Sullivan, P. M., and Maeda, N. (1999) Apo E structure determines VLDL clearance and atherosclerosis risk in mice. J Clin Invest 103:1579-86.
Lannert, H., and Hoyer, S. (1998) Intracerebroventricular administration of streptozotocin causes long-term diminutions in learning and memory abilities and in cerebral energy metabolism in adult rats. Behav Neurosci 112:199-208.
Loktionov A., Vorster H., O'Neill I. K., Nell T., Bingham S. A., Runswick S. A., Cummings J. H. (1999) Apolipoprotein E and methylenetetrahydrofolate reductase genetic polymorphisms in relation to other risk factors for cardiovascular disease in UK Caucasians and Black South Africans. Atherosclerosis 145:125-35.
Mattson, M. P. (1998). Experimental models of Alzheimer's Disease. Science and Medicine March/April: 16-25.
McKhann, G., Drachman, D., Folstein, M., Katzman, R., Price, D., and Stadlan, E. M. (1984). Clinical diagnosis of Alzheimer's disease: report of the NINCDS-ADRDA Work Group under the auspices of Department of Health and Human Services Task Force on Alzheimer's Disease. Neurology 34:939-44.
Meier-Ruge, W., Bertoni-Freddari, C., and Iwangoff, P. (1994) Changes in brain glucose metabolism as a key to the pathogenesis of Alzheimer's disease. Gerontology 40:246-52.
Messier, C., and Gagnon, M. (1996) Glucose regulation and cognitive functions: relation to Alzheimer's disease and diabetes. Behav Brain Res 75: 1-11.
Neve, R. L., and Robakis, N. K. (1998) Alzheimer's disease: a re-examination of the amyloid hypothesis. Trends Neurosci 21:15-9.
Nishimura, M., Yu, G., and St George-Hyslop, P. H. (1999) Biology of presenilins as causative molecules for Alzheimer disease. Clin Genet. 55:219-25.
Odle, J. (1997) New insights into the utilization of medium-chain triglycerides by the neonate: Observations from a pig model. J Nutr. 127:1061-7.
Reiman, E. M., Caselli, R. J., Yun, L. S., Chen, K., Bandy, D., Minoshima, S., Thibodeau, S, N., and Osborne, D. (1996) Preclinical evidence of Alzheimer's disease in persons homozygous for the epsilon 4 allele for apolipoprotein E. N Engl J Med 334:752-8.
Osuntokun B. O., Sahota A., Ogunniyi A. O., Gureje O., Baiyewu O., Adeyinka A., Oluwole S. O., Komolafe O., Hall K. S., Unverzagt F. W., et al (1995) Lack of an association between apolipoprotein E epsilon 4 and Alzheimer's disease in elderly Nigerians. Ann Neurol 38:463-5.
Roheim P. S., Carey M., Forte T., and Vega G. L. (1979) Apolipoproteins in human cerebrospinal fluid. Proc Natl Acad Sci USA 76:4646-9.
Schoonjans, K., J. Peinado-Onsurbe, et al. (1999). “3-Hydroxy-3-methylglutaryl CoA reductase inhibitors reduce serum triglyceride levels through modulation of apolipoprotein C-III and lipoprotein lipase.” FEBS Lett 452(3): 160-4.
Selkoe, D. J. (1994) Alzheimer's Disease: A central role for amyloid. J. Neruopathol. Exp. Neruol. 53:438-447.
Selkoe, D. J., (1999) Translating cell biology into therapeutic advances in Alzheimer's disease. Nature 399:A23-31.
Simpson, I. A., and Davies, P. (1994) Reduced glucose transporter concentrations in brains of patients with Alzheimer's disease: Ann Neurol 36:800-1.
Staels, B., J. Dallongeville, et al. (1998). “Mechanism of action of fibrates on lipid and lipoprotein metabolism.” Circulation 98(19): 2088-93.
Swaab, D. F., Lucassen, P. J., Salehi, A., Scherder, E. J., van Someren, E. J., and Verwer, R. W. (1998) Reduced neuronal activity and reactivation in Alzheimer's disease. Prog Brain Res 117:343-77.
Veech, Richard WO 98/41200. Sep. 24, 1998. Therapeutic Compositions
Veech, Richard WO 98/41201. Sep. 24, 1998. Therapeutic Compositions
Veech, Richard WO 00/15216. Mar. 23, 2000. Therapeutic Compositions (II)
Veneman, T., Mitrakou, A., Mokan, M., Cryer, P., and Gerich, J. (1994) Effect of hyperketonemia and hyperlacticacidemia on symptoms, cognitive dysfunction, and counterregulatory hormone responses during hypoglycemia in normal humans. Diabetes 43:1311-7.
Zekraoui L., Lagarde J. P., Raisonnier A., Gerard N., Aouizerate A., Lucotte G. (1997) High frequency of the apolipoprotein E*4 allele in African pygmies and most of the African populations in sub-Saharan Africa. Hum Biol 69:575-81.
Zubenko, G. S., Stiffler, J. S., Hughes, H. B., and Martinez, A. J. (1999) Reductions in brain phosphatidylinositol kinase activities in Alzheimer's disease. Biol Psychiatry 45:731-6.

### EXAMPLES

The following examples are offered by way of illustration and not by way of limitation.

### Example 1

### Nutritional Drink

Nutritional drinks are prepared using the following ingredients: emulsified MCT 100 g/drink, L-carnitine 1 gram/drink, mix of daily vitamins at recommended daily levels, and a variety of flavorings.

### Example 2

### Additional Formulations

Additional formulations can be in the form of Ready to Drink Beverages, Powdered Beverages, Nutritional drinks, Food Bars, and the like. Formulations for such are clear to those skilled in the art.

A. Ready to Drink Beverage. Ready to Drink Beverages are prepared using the following ingredients: emulsified MCT 5-100 g/drink, L-carnitine 250-1000 mg/drink, and a variety of flavorings and other ingredients used to increased palatability, stability, etc.

B. Powdered Beverages. MCT may be prepared in a dried form, useful for food bars and powdered beverage preparations. A powdered beverage may be formed from the following components: dried emulsified MCT 10-50 g, L-carnitine 250-500 mg, sucrose 8-15 g, maltodextrin 1-5 g, flavorings 0-1 g.

C. Food bar. A food bar would consist of: dried emulsified MCT 0.1-50 g, L-carnitine 250-500 mg, glycerin 1-5 g, corn syrup solids 5-25 g, cocoa 2-7 g, coating 15-25 g.

D. Gelatin Capsules. Hard or soft gelatin capsules are prepared using the following ingredients: MCT 0.1-1000 mg/capsule, L-carnitine 250-500 mg/capsule, Starch, NF 0-600 mg/capsule; Starch flowable powder 0-600 mg/capsule; Silicone fluid 350 centistokes 0-20 mg/capsule. The ingredients are mixed, passed through a sieve, and filled into capsules.

E. Tablets. Tablets are prepared using the following ingredients: MCT 0.1-1000 mg/tablet; L-carnitine 250-500 mg/tablet; Microcrystalline cellulose 20-300 mg/tablet; Starch 0-50 mg/tablet; Magnesium stearate or stearate acid 0-15 mg/tablet; Silicon dioxide, fumed 0-400 mg/tablet; silicon dioxide, colloidal 0-1 mg/tablet, and lactose 0-100 mg/tablet. The ingredients are blended and compressed to form tablets.

F. Suspensions. Suspensions are prepared using the following ingredients: 0.1-1000 mg MCT; 250-500 mg L-carnitine; Sodium carboxymethyl cellulose 50-700 mg/5 ml; Sodium benzoate 0-10 mg/5 ml; Purified water 5 ml; and flavor and color agents as needed.

G. Parenteral Solutions. A parenteral composition is prepared by stirring 1.5% by weight of MCT and L-carnitine in 10% by volume propylene glycol and water. The solution is made isotonic with sodium chloride and sterilized.

### Example 3

### Treating Alzheimer's Disease with Medium Chain Triglycerides

The purpose of this study was to explore whether hyperketonemia improves cognitive functioning in individuals with memory disorders. The goal of this trial was to test the hypothesis that sustained elevation of serum beta-hydroxybutyrate (BHB) levels through a large oral dose of medium chain triglycerides will improve memory and attention performances in individuals with Alzheimer's Disease and Mild Cognitive Impairment.

Participants

The sample consisted of 20 individuals with memory disorders recruited from Western Washington. Potential subjects were excluded if they had diabetes mellitus, hypoglycemia, major psychiatric disorders, or other major medical or neurological disorders such as hypertension, hypotension, cardiac problems, or COPD. In addition, patients were excluded from the study if they were taking medications with CNS effects, such as anti-psychotics, anti-anxiolytics, and anti-hypertensives. However, subjects were allowed to participate if they were taking anti-depressants. Four participants were taking anti-depressants at the time of the study.

Table 1 describes the demographics of the sample. Fifteen subjects met NINCIDS/ADRDA criteria for probable AD. The remaining 5 subjects were diagnosed with Mild Cognitive Impairment, believed to be a prodromal phase of AD. Participants ranged in age from 61 to 84 years of age (mean=74.7), and 25% of the sample was female. The sample was well educated with an average of 13.3 years of education. Ninety percent of the sample was Caucasian. Two non-Caucasian subjects were identified as African-American and American Indian. Participants were typically in the mild to moderate stages of dementia. The mean baseline MMSE was 22.2. Forty-seven percent of the participants had at least one apoE E-4 allele.

TABLE 1
Sample Demographics and Medical Information

| Variable | Mean | SD |
|---|---|---|
| Age | 74.7 | 6.7 |
| Education | 13.3 | 3.25 |
| BMI | 26.0 | 3.7 |
| MMSE | 22.2 | 5.5 |
| | n | Sample % |
| AD | 15 | 75 |
| MCI | 5 | 25 |
| Female | 5 | 25 |
| E4+ | 10/19 | 53 |
| Non-Caucasian | 2 | 10 |

Note:
SD = Standard Deviation, BMI = Body Mass Index, MMSE = Mini-Mental State Examination, E4+ = Subjects with at least one apoE E4 allele

Procedures

Subjects were recruited through medical clinics, senior centers, and ads in newspapers. Prospective subjects' medical histories and cognitive complaints were telephone screened by research nurses. Individuals were then referred to the Memory Disorders Clinic at the VA Puget Sound Health Care System (VAPSHCS) for clinical and/or neuropsychological evaluation. Routine laboratory assays and EKGs were completed to assist in diagnosis and determination of research inclusion.

The study was conducted with a randomized, double-blind placebo controlled, crossover design. Initially, subjects were asked to come to the VAPSHCS for three visits. During each visit, subjects received one of two conditions in a randomized order: emulsified long chain triglycerides as a placebo (232 ml of heavy whipping cream) or medium chain triglycerides (MCT; 40 ml of caprylic triglceride). caprylic triglyceride used in the study was NEOBEE 895 (obtained from Stepan, Inc.), comprising approximately 97% C8 fatty acids, with a specific gravity (at 25° C.) of 0.958, as the source of MCT. MCT were blended with 152 ml of heavy whipping cream. Vanilla and non-caloric sweetener were added to the drink for taste.

Subjects arrived in the morning after a 12-hour fast and blood was drawn to determine BHB levels and apoE genotyping (first visit only). Subjects then consumed the blended test sample described above. About ninety minutes later, a second blood draw occurred and a 30-minute cognitive testing session ensued. A final blood draw was then completed. Study visits were conducted at least one week apart, and not more than four weeks apart.

Neuropsychological Measures

Neuropsychological testing was performed by trained psychometrists using standardized procedures. A picture naming task, designed as a warm-up test, was completed at the beginning of the 30-minute test battery to reduce subject anxiety. The cognitive protocol included paragraph recall, the Stroop Color Word Interference Task, the Alzheimer's Disease Assessment Scale-Cognitive Subscale (ADAS-cog), and the Mini-Mental State Examination (MMSE).

The Logical Memory subtest of the Wechsler Memory Scale-III was used as the model for the paragraph recall test. Subjects heard brief narratives containing 25 bits of information. They were asked to recall as much information as possible, both immediately after hearing the story and again after a 10 minute delay.

The Stroop Color Word Interference Task is a test of selective attention. The first two conditions require speeded reading of color words and speeded naming of colored blocks on a page. In the third condition, color names are printed in discordant ink colors and subjects are asked to state the color of the ink while inhibiting reading of the color words. Total reading time was recorded.

The ADAS-cog is a mental status test designed specifically to rate the cognitive functioning of patients with Alzheimer's Disease. Scores range from 1 to 70 with higher scores indicating increased impairment.

The MMSE is a brief mental status test. Scores range from 0 to 30 with lower scores indicating increased impairment.

BHB Assays

Blood was processed immediately on the day of each subject's visit. Blood serum samples were kept in a −70° C. freezer until completion of the study. BHB levels were determined using a beta-hydroxybutyrate diagnostic kit (Sigma Diagnostics, Inc.). All samples were included in the assays and the lab was blinded to treatment conditions.

Results

Treatment Effects on BHB Levels

For BHB levels, a repeated measures ANCOVA was conducted with the apoE genotype as the independent factor (E4+ vs. E4−), and condition (treatment vs placebo) and time of blood draw (0, 90 min, and 120 min) as repeated factors and BMI as a covariate. BHB levels increased significantly with treatment (F[1,15]=5.16, p<0.039), and there was a significant difference in BHB levels at different time points (F[2, 14]=5.22, p<0.01). Significant increases in BHB levels were observed 90-minutes after treatment (p=0.007). In addition, there was a significant interaction between E4 status and time of blood draw (F[2, 14]=3.76, p=0.036). Contrasts revealed that the BHB levels for E4+ subjects continued to rise between the 90-minute and 120-minute blood draws in the treatment condition, while the BHB levels of E4− subjects held constant (p<0.003). Table 2 lists the BHB means and standard deviations for each E4 group.

TABLE 2
Mean BHB Values by Treatment Condition and apoE E4 Status

| | Placebo | | | | | |
|---|---|---|---|---|---|---|
| | Baseline | | 90′ | | 120′ | |
| E4 Status | Mean | SD | Mean | SD | Mean | SD |
| E4− | .04648 | .03565 | .07525 | .04780 | .09241 | .05803 |
| E4+ | .14013 | .17946 | .15589 | .16760 | .18549 | .18405 |
| | MCT Treatment | | | | | |
| | Baseline | | 90′ | | 120′ | |
| | Mean | SD | Mean | SD | Mean | SD |
| E4− | .04150 | .02375 | .53784 | .31535 | .51515 | .25437 |
| E4+ | .09504 | .08286 | .43022 | .18648 | .74142 | .37714 |

Note:
90′ = Values drawn 90 minutes after treatment; 120′ = Values drawn 120 minutes after treatment

Treatment Effects on Cognitive Performance

Repeated measures ANCOVAs were conducted with the apoE E4 allele as the independent factor (E4+ vs. E4−) and condition (treatment vs placebo) as the repeated factor, BHB levels at the time of cognitive testing as a covariate, and cognitive measures as the dependent variables. For the ADAS-cog, subjects without the apoE-E4 allele showed improvement following MCT administration, whereas E4+ subjects showed ADAS-cog Total Scores (lower scores indicate better performance) with slightly worse performance (table 2). This pattern resulted in a significant condition by E4 interaction (F[2, 14]=13.63, p=0.002).

The repeated measures ANCOVA with paragraph recall as the dependent measure revealed a trend interaction between the effects of treatment and BHB values measured just before testing (F[1, 14]=4.38, p=0.055). Subjects whose BHB levels were higher showed improved paragraph recall with MCT administration.

### Example 4

### Evaluation of oral MCT administered for up to 90 days in subjects with probable Alzheimer's disease of mild to moderate severity

In this example, a study was conducted to explore whether hyperketonemia improves cognitive functioning and memory in individuals with memory disorders, such as Alzheimer's disease. The goal of this trial was to test the hypothesis that sustained elevation of serum beta-hydroxybutyrate (βHB) levels through a large oral dose of medium chain triglycerides (MCT) will improve memory and attention performances in individuals with age associated cognitive decline or a dementing illness such as Alzheimer's disease or Mild Cognitive Impairment. The study was a randomized, double-blind, placebo-controlled, parallel, multi-center design. The subjects received either oral medium chain triglycerides (MCT) or placebo for ninety days followed by a two week washout period.

MCT or matching placebo was administered once a day for ninety days by mixing powder in one glass (approximately 8 oz.) of a liquid (i.e., water, juice, milk). For the first seven days of treatment, the subjects ingested 30 grams of powder (approximately 10 grams of Medium Chain Triglycerides) or placebo QD, increasing the dose to 60 gram QD (approximately 20 gram MCT) on Day 8 through Day 90. Following the end of the ninety-day dosing period, subjects had a two week washout period. The MCT treatment in this study was a 40 gm dose containing 50% caprylic triglceride (equivalent to 20 g of MCT). The caprylic triglyceride used in the study was NEOBEE 895 (obtained from Stepan, Inc.), comprising approximately 97% C8 fatty acids.

Efficacy outcome measures were: a) Alzheimer's Disease Assessment Scale-Cognitive Subscale (ADAS-Cog), b) Alzheimer's Disease Cooperative Study-Clinician's Global Impression of Change (ADCS-CGIC) and c) Mini-Mental State Exam (MMSE).

The Alzheimer's Disease Assessments Scale—Cognitive Subscale (ADAS-Cog) (Rosen et al. Am J Psychiatry 1984; 141(11): 1356-1364) is designed to measure cognitive symptom change in subjects with Alzheimer's disease. The standard 11 items are word-list recall, naming, commands, constructional praxis, ideational praxis, orientation, word recognition, spoken language ability, comprehension of spoken language, word-finding difficulty, and remembering test instructions.

The Alzheimer's Disease Cooperative Study—Clinician's Global Impression of Change (ADCS-CGIC) (Schneider et al. Alzheimer Disease and Associated Disorders 1997; 11(Suppl. 2) S22-S32) was used to assess change from the Baseline in the clinician's impression of change.

The Mini-Mental State Exam (MMSE) (Folstein et al. J. Psychia Res 1975; 12:189-198) was used as an assessment of mental status in five domains: orientation, registration, attention, recall and language.

Each subject was seen five times: at screening, at baseline, and at post baseline days 45, 90, and 104. At Visit 1 (screen), the following assessments were performed: demographics, medical/surgical history, NINCDS-ADRDA criteria, DSM-IV criteria, Modified Hachiniski Ischemia Scale, prior and concomitant medications, physical examinations, height, weight, vital signs, CT scan/MRI (performed if not previously done in last 18 months), ECG, TSH, B12, βHB serum level, safety laboratory assessments, ADAS-Cog, MMSE and Cornell Scale for Depression in Dementia.

Visit 2 (Baseline) occurred within 4 weeks (28 days) of Visit 1. The following assessments were conducted: adverse events (since initiation of Screen), concomitant medications, vital signs, ADAS-Cog, ADCS-CGIC and MMSE. Following completion of those assessments, eligible subjects were randomized, and the first dose (30 gm) of study medication was administered to the subject.

Visit 3 occurred 45 days (±3 days) after the Baseline visit. The following assessments were performed: adverse events, concomitant medications, vital signs, ADAS-Cog, ADCS-CGIC and MMSE. A blood sample was taken for serum βHB levels prior to dosing and 2 hr post-dosing.

Visit 4 occurred 90 days (±3 days) after the Baseline visit. The following assessments were performed: adverse events, concomitant medications, vital signs, ADAS-Cog, ADCS-CGIC, and MMSE. A blood sample was taken for serum βHB levels prior to dosing and 2 hr post-dosing.

Visit 5 occurred 104 days (±3 days) after the Baseline visit. The following assessments were performed: adverse events, concomitant medications, vital signs, weight, physical examination, ECG, safety labs, ADAS-Cog, ADCS-CGIC, and MMSE. A final blood sample was taken for serum βHB levels.

Change from Baseline at Day 90 was considered the primary measure of efficacy. Treatment comparisons for ADAS-Cog and MMSE (secondary outcome) were tested using ANCOVA with Treatment and Center as Factors and Age and Baseline scores as covariates. Treatment comparisons for ADCS-CGIC were done using Cochran-Mantel-Haenszel Tests. Treatment by genotype comparisons were done using a 2 way ANOVA with Treatment and ApoE4 status as variables. All comparisons used intent to treat populations (ITT) with last observation carried forward (LOCF).

Results: ADAS-Cog. For all patients, when comparing MCTs and Placebo for change at Day 90 from Baseline, treatment with MCTs led to a decline of 0.26 points of total ADAS-Cog, whereas the Placebo group showed a 1.93 point decline, indicating that the MCT-treated patients showed lessened decline of cognitive function than the Placebo patients.

When comparing MCT and Placebo for change at Day 90 from Baseline for ApoE ε4(−) patients, the ApoE ε4(−) subjects improved cognitively (−1.75 points) on their ADAS-Cog scores, whereas the ApoE ε4(−) subjects on placebo declined (1.61 points) on their ADAS-Cog score. Scores on ADAS-Cog are inversely related to cognitive function. Therefore, lower scores represent improved performance on tests of memory, cognition, etc. The change in ADAS-Cog scores between MCT group and Placebo group was 3.36 points. Through the course of the study, subjects treated with MCTs generally showed improvement in cognition via their ADAS-Cog scores.

AD Cooperative Study-Clinical Global Impression of Change (ADCS-CGIS). As for ADAS-Cog, lower scores indicate improved performance. After 90 days of treatment, ApoE ε4(−) subjects on MCTs scored an average of 4.17 points, whereas the ApoE ε4(−) subjects on Placebo scored an average of 4.68 points, showing decreased decline in the MCT patients. Therefore, improved scores were found in ApoE ε4(−) subjects treated with MCT.

Through the course of the study, subjects treated with MCTs generally showed lowered scores on CGIC, indicating decrease in decline compared with Placebo, ApoE ε4(−) subjects showed lowered CGIC scores at Day 45 and Day 90.

As discussed herein in the present Example, levels of β-hydroxybutyrate (βHB, a ketone body) were determined for patients in the study. It was found that there was a significant pharmacologic response between βHB plasma levels and ADAS-Cog scores in ApoE ε4(−) patients. Data shows a correlation between change in ADAS-Cog from Baseline to Day 90 and serum Cmax βHB levels.

Concomitant AD Medications

Over 80% of the subjects in this trial were taking stable doses of AD medications at study entry. This is important in that the efficacy observed with MCT is “on top of” any improvements provided by these medications. It also demonstrates that MCT administration is compatible with the use of these other AD medications and MCT could be administered in combination with these agents. Furthermore the analysis of the data (as provided below) indicates that use of MCT in conjunction with these agents can provide improvement in cognitive performance compared to their use separately.

The number and percent of subjects taking medications for the treatment of AD during the course of study were analyzed for both treatment groups. Per the study protocol, subjects taking cholinesterase inhibitors (Aricept®, Exelon®, or Reminyl/Razadyne®) or NMDA receptor antagonists (Namenda®) were permitted to enroll in the study as long as their dosing regimen had been stable for at least 3 months prior to study enrollment. The proportion of subjects within each treatment group who were taking one or more of these medications is presented below (Table 3). Among the AD medications used in this study, Aricept was more frequently taken by MCT subjects and Namenda® was taken more frequently by Placebo subjects.

TABLE 3
Concomitant Medications Specifically for AD By Treatment Group*

| MCT | | | | Placebo | | | |
|---|---|---|---|---|---|---|---|
| N = 86 | | | | N = 66 | | | |
| | | | Reminyl ®/ | | | | Reminyl ®/ |
| Aricept ® | Namenda ® | Exelon ® | Razadyne ® | Aricept ® | Namenda ® | Exelon ® | Razadyne ® |
| n (%) | n (%) | n (%) | n (%) | n (%) | n (%) | n (%) | n (%) |
| 43 (50.0%) | 32 (37.2%) | 11 (12.8%) | 3 (03.5%) | 28 (42.4%) | 31 (47.0%) | 11 (16.7%) | 9 (13.6%) |

Program source: lconmed.sas + Listing 32.1
*Note:
subjects may have taken more than one AD medication

Overall, the proportion of subjects taking one or more AD medications during the course of study was slightly higher in the Placebo group than in the MCT group (Table 4). Sixty-six of 86 (78.7%) MCT subjects and 55 of 66 Placebo subjects (83.3%) were taking AD medications. In addition, the proportion of subjects taking more than one AD medication was also higher among Placebo subjects than among subjects treated with MCT (24 of 86 [27.9%] Ketasyn; 24 of 66 [36.4%] Placebo).

TABLE 4
Number (%) of Subjects Taking One or More
AD Medications By Treatment Group

| | MCT | Placebo |
|---|---|---|
| | N = 86 | N = 66 |
| n (%) of subjects taking | 66 (76.7%) | 55 (83.3%) |
| AD medications | | |
| n (%) of subjects taking | 23 (2337%) | 24 (36.4%) |
| >1 AD medication | | |

Program source: lconmed.sas + Listing 32.2 & 32.3 pts AD meds

A two-way ANOVA was carried out to evaluate change from baseline to Day 90 in ADAS-Cog scores across treatment groups for subjects taking AD medications (Table 5).

TABLE 5
Concomitant Medication Effects on Change in ADAS-Cog Scores* (ITT Population) N = 140

| Statistic | Aricept ™ | Namenda ™ | Exelon ™ | Reminyl/Razadyne ™ |
|---|---|---|---|---|
| N for Treatment (Placebo, MCT) | 63, 77 | 63, 77 | 63, 77 | 63, 77 |
| N for AD Medication (N, Y) | 78, 62 | 83, 57 | 119, 21 | 128, 12 |
| N for Treatment* AD Medication | 37, 26, 41, 36 | 34, 29, 49, 28 | 52, 11, 67, 10 | 54, 9.74, 3 |
| (PN, PY, KN, KY) | | | | |
| LSMEANS for Treatment (P, K) | 1.090033, −0.31978 | 1.32000, −0.03827 | 1.62491, 0.87015 | 1.56451, −3.04054 |
| LSMEANS for AD Medication (N, Y) | 0.83893, −0.06838 | −0.44639, 1.72812 | 0.14355, 2.35152 | 0.50545, −1.98148 |
| LSMEANS for Treatment*AD | 1.87297, 0.30769, | 0.14804, 2.49195, | 1.01346, 2.23636, | 1.09198, 2.03704, |
| Medication (PN, PY, KN, KY) | −0.19512, −0.44444 | −1.04082, 0.96429 | −0.72637, 2.46667 | −0.08108, −6.00000 |

*Change from Baseline (Day 90-Day 0)
# 2 way ANOVA calculated using PROC GLM Type 3 SS. P-values of differences of the type 3 SS LSMEANS
SAS Program Name: Intable1trtari.sas Date:27DEC2006:12:31:41 Source Data: &source

The results in Table 5 show that in each case the ADAS-Cog score for each of the AD drugs the score is better (LSMEANS for Treatment) (note that a lower or negative number constitutes improvement) when the patients are taking MCT in addition to their AD medication.

After 90 days on-study, MCT subjects who were taking Aricept® or Reminyl®/Razadyne® demonstrated measurable improvements from Baseline in mean ADAS-Cog scores when compared to subjects taking AD medications and Placebo (mean change of −0.44444 and −6.00000, respectively). For Aricept, there was an average improvement between MCT and Placebo of 0.75 points in ADAS-Cog. For Reminyl/Razadyne™, there was an average improvement between MCT and Placebo of 8.04 points in ADAS-Cog. For Namenda®, there was an average improvement between MCT and Placebo of 1.53 points in ADAS-Cog.

The present Example demonstrates that MCT administration can be used in conjunction with the use of these other AD medications and MCT could be administered in combination with these agents. Therefore a combination of MCT and one or more of the AD medications is a preferred embodiment of this invention. Furthermore, the analysis provided above indicates that there is a benefit arising from the co-administration of MCT and either ARICEPT, NAMENDA or REMINYL.

Further support for the use of a combination of MCT with currently prescribed AD medications is found in the analysis of subjects at Day 45. Subjects taking any AD medication and MCT show a statistically significant improvement in ADAS-Cog scores at Day 45 (mean improvement of 1.98 points relative to Placebo, p-value 0.0443, see Table 6).

TABLE 6
Change in ADAS-Cog at Day 45

| | | | | |
|---|---|---|---|---|
| Level | Number | Mean | Std Error | p-value |

Any AD medication

| | | | | |
|---|---|---|---|---|
| MCT | 58 | −0.0402 | 0.67351 | |
| Placebo | 53 | 1.9428 | 0.70457 | 0.0443 |

Reminyl/Razadyne

| | | | | |
|---|---|---|---|---|
| MCT | 3 | −3.5556 | 2.0628 | |
| Placebo | 9 | 1.5185 | 1.1910 | 0.0590 |

MCTs are converted in the liver to ketone bodies, such as βHB, acetoacetate and acetone. Ketone bodies can be used as a metabolic substrate for a variety of cell types and as demonstrated herein in the present Example, the higher the level of serum ketone body βHB, the greater improvement seen in ADAS-Cog in ApoE ε4− subjects, strongly confirming the beneficial effects of daily MCT administration.

These results show that for Alzheimer's disease patients MCT treatment in combination with current AD medications leads to improved cognition or memory in as little as 45 days.

### Example 5

### Formulations

Boost™ with fiber nutritional beverage (Mead Johnson Nutritionals) and similar products such as Ensure™ have the following general aspects and ingredients. Amounts are per 8 fl. oz. container, which is planned to provide 20-25% of the daily requirements. Tailoring the following formulation for use in subjects with Alzheimer's disease or mild cognitive impairment would be very beneficial.

Calories, kcal 250

Calories from fat 70

Protein, g 11

Fat, g 8

Saturated fat, g 1.5

Carbohydrate, g 33

Dietary Fiber, g 3

Sugars, g 16

Water, g 200

Vitamin A, IU 830

Vitamin D, IU 100

Vitamin E, IU 5

Vitamin K, .mcg 23

Vitamin C, mg 30

Folic Acid, .mcg 100

Thiamin, mg 0.37

Riboflavin, mg 0.43

Niacin, mg 5

Vitamin B6, mg 0.5

Vitamin B12, .mcg 1.5

Biotin, .mcg 75

Pantothenic Acid, mg 2.5

Calcium, mg 200

Phosphorus, mg 167

Iodine, μg 25

Iron, mg 3

Magnesium, mg 67

Copper, mg 0.33

Zinc, mg 3.3

Manganese, mg 0.42

Chloride, mg 330

Potassium, mg 330

Sodium, mg 170

The present invention describes a novel formulation wherein the above formula is supplemented with about 1 to 80 grams for medium chain triglycerides and about 10 to 2000 mg of L-carnitine or acetyl-L-carnitine. Alternatively, more preferably, 5 to 50 grams of medium chain triglycerides and about 50 to 1000 mg of L-carnitine or acetyl-L-carnitine. Alternatively, more preferably, 10 to 30 grams of medium chain triglycerides and about 100 to 500 mg of L-carnitine or acetyl-L-carnitine.

### Example 6

### Formulations

Boost™. High Protein Powder (Mead Johnson Nutritionals) or similar products are high-protein, low-fat nutritional powders that can be mixed with skim milk or water. About 54 g of the powder is to be mixed with 8 fluid ounces (fl. oz) of water, and is said to provide at least 25% of the US RDA of most essential vitamins and minerals in 200 calories. It has virtually no fat. When mixed with skim milk, the mixture provides about 290 calories and about 33% of the US RDA of most essential vitamins and minerals. Tailoring the following formulation for use in subjects with Alzheimer's disease or mild cognitive impairment would be very beneficial.

The water mixture provides the following:

Protein, g 13

Carbohydrate, g 36

Sugars, g 35

Water, g 240

Vitamin A, IU 1290

Vitamin D, IU 33

Vitamin E, IU 10

Vitamin C, mg 20

Folic Acid, .mcg 133

Thiamin, mg 0.4

Riboflavin, mg 0.2

Niacin, mg 6.8

Vitamin B6, mg 0.55

Vitamin B12, .mcg 1

Biotin, .mcg 93

Pantothenic Acid, mg 2.7

Calcium, mg 290

Phosphorus, mg 250

Iodine, .mcg 40

Iron, mg 6

Magnesium, mg 105

Copper, mg 0.7

Zinc, mg 4

Manganese, mg 1

Chloride, mg 220

Potassium, mg 560

Sodium, mg 189

The present invention describes a novel formulation wherein the above formula is supplemented with about 1 to 80 grams for medium chain triglycerides and about 10 to 2000 mg of L-carnitine or acetyl-L-carnitine. Alternatively, more preferably, 5 to 50 grams of medium chain triglycerides and about 50 to 1000 mg of L-carnitine or acetyl-L-carnitine. Alternatively, more preferably, 10 to 30 grams of medium chain triglycerides and about 100 to 500 mg of L-carnitine or acetyl-L-carnitine.

### Example 7

### Formulations

Boost™ Pudding (Mead Johnson) or similar products are labeled for intended use in geriatric patients, malnourished cancer patients and persons desiring weight control. The current formulation provides 240 calories in 5 ounces, low sodium and cholesterol, and 15-20% of the US RDA requirements for most vitamins and minerals. Tailoring the following formulation for use in subjects with Alzheimer's disease or mild cognitive impairment would be very beneficial.

Protein, g 7

Fat, g 9

Saturated Fat, g 1.5

Sugars, g 27

Water, g 92

Vitamin A, IU 750

Vitamin D, IU 60

Vitamin E, IU 4.5

Vitamin C, mg 9

Folic Acid, .mcg 60

Thiamin, mg 0.23

Riboflavin, mg 0.26

Niacin, mg 3

Vitamin B6, .mcg 300

Vitamin B12, .mcg 0.9

Biotin, .mcg 45

Pantothenic Acid, mg 1.5

Calcium, mg 220

Phosphorus, mg 220

Iodine, .mcg 23

Iron, mg 2.7

Magnesium, mg 60

Copper, mg 0.3

Zinc, mg 2.3

Chloride, mg 200

Potassium, mg 320

Sodium, mg 120

The present invention describes a novel formulation wherein the above formula is supplemented with about 1 to 80 grams for medium chain triglycerides and about 10 to 2000 mg of L-carnitine or acetyl-L-carnitine. Alternatively, more preferably, 5 to 50 grams of medium chain triglycerides and about 50 to 1000 mg of L-carnitine or acetyl-L-carnitine. Alternatively, more preferably, 10 to 30 grams of medium chain triglycerides and about 100 to 500 mg of L-carnitine or acetyl-L-carnitine.

### Example 8

### Formulations

Nutritional bars have been developed for a variety of diets and activity levels (e.g., LUNA., from Clif Bar, Inc., Berkeley, Calif.) but have no effect on cognitive performance. An example of such a nutritional bar is shown below. Percents are the portion of minimum daily requirements. Tailoring the following formulation for use in subjects with Alzheimer's disease or mild cognitive impairment would be very beneficial.

Total Fat, g 4

Saturated Fat, g 3

Sodium, mg 50

Potassium, mg 90

Total Carbohydrate, g 26

Dietary Fiber, g 1

Sugars, g 15

Other Carbs, g 10

Protein, g 10

Vitamin A, % 25

Vitamin C, % 100

Calcium, % 35

Iron, % 35

Vitamin K, % 100

Thiamin, % 100

Riboflavin, % 100

Niacin, % 100

Vitamin B6, % 100

Folic Acid, % 100

Vitamin B12, % 100

Biotin, % 100

Pantothenic Acid, % 100

Phosphorus, % 35

Iodine, % 35

Zinc, % 35

Selenium, % 35

Copper, % 35

Manganese, % 35

Chromium, % 35

Molybdenum, % 35

The present invention describes a novel formulation wherein the above formula is supplemented with about 1 to 80 grams for medium chain triglycerides and about 10 to 2000 mg of L-carnitine or acetyl-L-carnitine. Alternatively, more preferably, 5 to 50 grams of medium chain triglycerides and about 50 to 1000 mg of L-carnitine or acetyl-L-carnitine. Alternatively, more preferably, 10 to 30 grams of medium chain triglycerides and about 100 to 500 mg of L-carnitine or acetyl-L-carnitine.

### Example 9

### Formulations

A formulation of flavored gelatin (e.g., JELL-O™) provides 130 calories in 227 g. Tailoring the following formulation for use in active elders would be highly beneficial. Percents are the portion of minimum daily requirements.

Protein, g 2

Fat, g 0

Saturated Fat, g 0

Sugars, g 31

Vitamin A, % 6

Vitamin C, % 4

Calcium, % 0

Iron, % 2

Sodium, mg 75

Gelatin flavors can include: apricot, berry blue, black cherry, cherry, cranberry, cranberry raspberry, cranberry strawberry, grape, lemon, lime, mandarin orange, mango, mixed fruit, orange, peach, peach passion fruit, island pineapple, raspberry, strawberry, strawberry banana, strawberry kiwi, watermelon, wild berry, and wild strawberry, among others.

The present invention describes a novel formulation wherein the above formula is supplemented with about 1 to 80 grams for medium chain triglycerides and about 10 to 2000 mg of L-carnitine or acetyl-L-carnitine. Alternatively, more preferably, 5 to 50 grams of medium chain triglycerides and about 50 to 1000 mg of L-carnitine or acetyl-L-carnitine. Alternatively, more preferably, 10 to 30 grams of medium chain triglycerides and about 100 to 500 mg of L-carnitine or acetyl-L-carnitine.

All publications and patent applications cited in this specification are herein incorporated by reference as if each individual publication or patent application were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A composition for the treatment of or prevention of Alzheimer's disease or mild cognitive impairment, comprising:
i) medium chain triglycerides (MCT) of the formula: 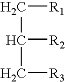
wherein the R1, R2, and R3 esterified to the glycerol backbone are each independently fatty acids having 5-12 carbon chains in an amount effective for the treatment of or prevention of loss of cognitive function in a mammal caused by reduced neuronal metabolism in dementia of Alzheimer's type or mild cognitive impairment; and
ii) a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof.

2. The composition of claim 1, wherein greater than 95% of the R1, R2, and R3 carbon chains are 8 carbons in length.

3. The composition of claim 1, wherein the composition further comprises glucose.

4. The composition of claim 1, wherein the therapeutic agent is an anti-Alzheimer's agent.

5. The composition of claim 4, wherein the anti-Alzheimer's agent is selected from the group consisting of modulators of cholinesterase, acetylcholine synthesis modulators, acetylcholine storage modulators, acetylcholine release modulators, NMDA receptor antagonists, beta-amyloid inhibitors, β-amyloid plaque removal agents (including vaccines), inhibitors of β-amyloid plaque formation, amyloid precursor protein processing enzyme inhibitors, β-amyloid converting enzyme (BACE) inhibitors, β-secretase inhibitors, γ-secretase modulators, nerve growth factor agonists, hormone receptor blockade agents, neurotransmission modulators, anti-inflammatory agents, and combinations thereof.

6. The composition of claim 5, wherein the anti-Alzheimer's agent is a modulator of cholinesterase.

7. The composition of claim 6, wherein the modulator of cholinesterase is selected from the group consisting of tacrine, donepezil, rivastigmine, galantamine, physostigmine, neostigmine, Huperzine A, icopezil, 4-[(5,6-dimethoxy-2-fluoro-1-indanon)-2-yl]methyl-1-(3-fluorobenzyl)piperidine hydrochloride), zanapezil, metrifonate, (n-(4-acetyl-1-piperazinyl)-p-fluorobenzamide-hydrate), N-methyl-N2-pyropinyldibenz[b,f]oxepine-10-methanamine), (S)-α-amino-5-(phosphonomethyl)-[1,1′-biphenyl]-3-propionic acid, and combinations thereof.

8. The composition of claim 5, wherein the anti-Alzheimer's agent is an NMDA receptor antagonist.

9. The composition of claim 8, wherein the NMDA receptor antagonist is selected from the group consisting of memantine, neramexane (1,3,3,5,5-pentamethylcyclohexan-1-amine), and combinations thereof.

10. The composition of claim 5, wherein the anti-Alzheimer's agent is selected from the group consisting of tacrine, donepezil, rivastigmine, galantamine, physostigmine, neostigmine, icopezil (5,7-dihydro-3-[2-[1-(phenylmethyl)-4-piperidinyl]ethyl]-6H-pyrrolo-[4,5-f]-1,2-benzisoxazol-6-one maleate), 4-[(5,6-dimethoxy-2-fluoro-1-indanon)-2-yl]methyl-1-(3-fluorobenzyl)piperidine hydrochloride), zanapezil, metrifonate, N-(4-acetyl-1-piperazinyl)-p-fluorobenzamide-hydrate, N-methyl-N2-pyropinyldibenz[b,f]oxepine-10-methanamine, (S)-α-amino-5-(phosphonomethyl)-[1,1′-biphenyl]-3-propionic acid), memantine, 1,3,3,5,5-pentamethylcyclohexan-1-amine, tarenflurbil, tramiprosate, clioquinol, 1-(2-(2-Naphthyl)ethyl)-4-(3-trifluoromethylphenyl)-1,2,3,6-tetrahydropyridine, Huperzine A, posatirelin, leuprolide, ispronicline, (3-aminopropyl)(n-butyl)phosphinic acid (SGS-742), N-methyl-5-(3-(5-isopropoxypyridinyl))-4-penten-2-amine (ispronicline), 1-decanaminium, N-(2-hydroxy-3-sulfopropyl)-N-methyl-N-octyl-, salicylates, aspirin, amoxiprin, benorilate, choline magnesium salicylate, diflunisal, faislamine, methyl salicylate, magnesium salicylate, salicyl salicylate, diclofenac, aceclofenac, acemetacin, bromfenac, etodolac, indometacin, nabumetone, sulindac, tolmetin, ibuprofen, carprofen, fenbufen, fenoprofen, flurbiprofen, ketoprofen, ketorolac, loxoprofen, naproxen, tiaprofenic acid, suprofen, mefenamic acid, meclofenamic acid, phenylbutazone, azapropazone, metamizole, oxyphenbutazone, sulfinprazone, piroxicam, lornoxicam, meloxicam, tenoxicam, celecoxib, etoricoxib, lumiracoxib, parecoxib, rofecoxib, valdecoxib, nimesulide, arylalkanoic acids, 2-arylpropionic acids (profens), N-arylanthranilic acids (fenamic acids), pyrazolidine derivatives, oxicams, COX-2 inhibitors, sulphonanilides, essential fatty acids, minozac (2-(4-(4-methyl-6-phenylpyridazin-3-yl)piperazin-1-yl)pyrimidine dihydrochloride hydrate), and combinations thereof.

11. The composition of claim 1, wherein the MCT is in an amount effective to induce hyperketonemia.

12. The composition of claim 11, wherein the hyperketonemia comprises a rise in circulating β-hydroxybutyrate in a patient to between about 0.1 millimolar to about 10 millimolar at about two hours post administration.

13. The composition of claim 1 wherein the MCT is administered at a dose of about 0.05 g/kg/day to about 10 g/kg/day.

14. The composition of claim 1, wherein the composition is a ready-to-drink beverage, powdered beverage formulation, nutritional or dietary supplement selected from the group consisting of gelatin capsule or tablet, suspension, parenteral solution, or a food product formulated for human consumption.

15. A method of treating dementia of Alzheimer's type or mild cognitive impairment, comprising the steps of:
(a) identifying a mammal having, or at risk of dementia of Alzheimer's type or mild cognitive impairment;
(b) administering to the mammal a first composition comprising medium chain triglycerides (MCT) of the formula: 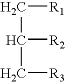
wherein the R1, R2, and R3 esterified to the glycerol backbone are each independently fatty acids having 5-12 carbon chains in an amount effective for the treatment of or prevention of loss of cognitive function caused by reduced neuronal metabolism in dementia of Alzheimer's type or mild cognitive impairment; and
(c) administering to the mammal a second composition comprising a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof.

16. The method of claim 15 wherein greater than 95% of the R1, R2, and R3 carbon chains are 8 carbons in length.

17. The method of claim 15, wherein the composition comprising MCT further comprises glucose.

18. The method of claim 15, wherein the MCT is administered in an amount effective to induce hyperketonemia.

19. The method of claim 18, wherein the hyperketonemia comprises a rise in circulating β-hydroxybutyrate in the mammal to between about 0.1 millimolar to about 10 millimolar at about two hours post administration.

20. The method of claim 15 wherein the composition comprising MCT is administered at a dose of MCT of about 0.05 g/kg/day to about 10 g/kg/day.

21. The method of claim 15, wherein the composition comprising MCT is administered as part of a daily treatment regimen for at least about three months.

22. The method of claim 15, comprising the further step of determining the ApoE status of the mammal and selecting a mammal for treatment if the mammal is ApoE4(−).

23. The method of claim 15, wherein efficacy for treatment of or prevention of loss of cognitive function caused by reduced neuronal metabolism in dementia of Alzheimer's type or mild cognitive impairment is determined by results from at least one neuropsychological test.

24. The method of claim 15, wherein the therapeutic agent is an anti-Alzheimer's agent.

25. The method of claim 24, wherein the anti-Alzheimer's agent is selected from the group consisting of modulators of cholinesterase, acetylcholine synthesis modulators, acetylcholine storage modulators, acetylcholine release modulators, NMDA receptor antagonists, beta-amyloid inhibitors, β-amyloid plaque removal agents (including vaccines), inhibitors of β-amyloid plaque formation, amyloid precursor protein processing enzyme inhibitors, β-amyloid converting enzyme (BACE) inhibitors, β-secretase inhibitors, γ-secretase modulators, nerve growth factor agonists, hormone receptor blockade agents, neurotransmission modulators, anti-inflammatory agents, and combinations thereof.

26. The method of claim 24, wherein the anti-Alzheimer's agent is a modulator of cholinesterase.

27. The method of claim 26, wherein the modulator of cholinesterase is selected from the group consisting of tacrine, donepezil, rivastigmine, galantamine, physostigmine, neostigmine, Huperzine A, icopezil, 4-[(5,6-dimethoxy-2-fluoro-1-indanon)-2-yl]methyl-1-(3-fluorobenzyl)piperidine hydrochloride), zanapezil, metrifonate, (n-(4-acetyl-1-piperazinyl)-p-fluorobenzamide-hydrate), N-methyl-N2-pyropinyldibenz[b,f]oxepine-10-methanamine), (S)-α-amino-5-(phosphonomethyl)-[1,1′-biphenyl]-3-propionic acid, and combinations thereof.

28. The method of claim 24, wherein the anti-Alzheimer's agent is an NMDA receptor antagonist.

29. The method of claim 28, wherein the NMDA receptor antagonist is selected from the group consisting of memantine, neramexane (1,3,3,5,5-pentamethylcyclohexan-1-amine), and combinations thereof.

30. The method of claim 15, wherein the therapeutic agent is selected from the group consisting of tacrine, donepezil, rivastigmine, galantamine, physostigmine, neostigmine, icopezil (5,7-dihydro-3-[2-[1-(phenylmethyl)-4-piperidinyl]ethyl]-6H-pyrrolo-[4,5-f]-1,2-benzisoxazol-6-one maleate), 4-[(5,6-dimethoxy-2-fluoro-1-indanon)-2-yl]methyl-1-(3-fluorobenzyl)piperidine hydrochloride), zanapezil, metrifonate, N-(4-acetyl-1-piperazinyl)-p-fluorobenzamide-hydrate, N-methyl-N2-pyropinyldibenz[b,f]oxepine-10-methanamine, (S)-α-amino-5-(phosphonomethyl)-[1,1′-biphenyl]-3-propionic acid), memantine, 1,3,3,5,5-pentamethylcyclohexan-1-amine, tarenflurbil, tramiprosate, clioquinol, 1-(2-(2-Naphthyl)ethyl)-4-(3-trifluoromethylphenyl)-1,2,3,6-tetrahydropyridine, huperzine A, posatirelin, leuprolide, ispronicline, (3-aminopropyl)(n-butyl)phosphinic acid (SGS-742), N-methyl-5-(3-(5-isopropoxypyridinyl))-4-penten-2-amine (ispronicline), 1-decanaminium, N-(2-hydroxy-3-sulfopropyl)-N-methyl-N-octyl-, salicylates, aspirin, amoxiprin, benorilate, choline magnesium salicylate, diflunisal, faislamine, methyl salicylate, magnesium salicylate, salicyl salicylate, diclofenac, aceclofenac, acemetacin, bromfenac, etodolac, indometacin, nabumetone, sulindac, tolmetin, ibuprofen, carprofen, fenbufen, fenoprofen, flurbiprofen, ketoprofen, ketorolac, loxoprofen, naproxen, tiaprofenic acid, suprofen, mefenamic acid, meclofenamic acid, phenylbutazone, azapropazone, metamizole, oxyphenbutazone, sulfinprazone, piroxicam, lornoxicam, meloxicam, tenoxicam, celecoxib, etoricoxib, lumiracoxib, parecoxib, rofecoxib, valdecoxib, nimesulide, arylalkanoic acids, 2-arylpropionic acids (profens), N-arylanthranilic acids (fenamic acids), pyrazolidine derivatives, oxicams, COX-2 inhibitors, sulphonanilides, essential fatty acids, minozac (2-(4-(4-methyl-6-phenylpyridazin-3-yl)piperazin-1-yl)pyrimidine dihydrochloride hydrate), and combinations thereof.

31. A liquid dosage form for oral consumption comprising:
i) a unit dose of MCT sufficient to a) raise blood levels of D-β-hydroxybutyrate to about 0.1 to about 5 mM or b) raise urinary excretion levels of D-β-hydroxybutyrate to about 5 mg/dL to about 160 mg/dL; a plurality of vitamins; flavoring, and a carbohydrate source and wherein the MCT are of the formula: 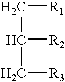
wherein the R1, R2, and R3 esterified to the glycerol backbone are each independently fatty acids having carbon chains of 5-12 carbons; and
ii) a therapeutic agent selected from the group consisting of anti-Alzheimer's agents, anti-diabetic agents, agents capable of increasing utilization of lipids, anti-atherosclerotic agents, anti-hypertensive agents, anti-inflammatory agents, anti-obesity agents, and combinations thereof.
